SEQUENCE LISTING (30 sequences)

SEQ ID NO: 1 - DNA, 1258 bp, Lactobacillus plantarum
  misc_feature (2)..(4): n is a, c, g, or t
  misc_feature (6)..(6): n is a, c, g, or t
  misc_feature (12)..(12): n is a, c, g, or t
  misc_feature (128)..(128): n is a, c, g, or t
  misc_feature (1010)..(1010): n is a, c, g, or t
  misc_feature (1039)..(1039): n is a, c, g, or t
  misc_feature (1043)..(1043): n is a, c, g, or t
  misc_feature (1082)..(1082): n is a, c, g, or t
  misc_feature (1087)..(1087): n is a, c, g, or t
  misc_feature (1089)..(1089): n is a, c, g, or t
  misc_feature (1104)..(1104): n is a, c, g, or t
  misc_feature (1109)..(1109): n is a, c, g, or t
  misc_feature (1112)..(1113): n is a, c, g, or t
  misc_feature (1115)..(1115): n is a, c, g, or t
  misc_feature (1119)..(1119): n is a, c, g, or t
  misc_feature (1125)..(1125): n is a, c, g, or t
  misc_feature (1136)..(1143): n is a, c, g, or t
  misc_feature (1148)..(1149): n is a, c, g, or t
  misc_feature (1151)..(1151): n is a, c, g, or t
  misc_feature (1155)..(1159): n is a, c, g, or t
  misc_feature (1161)..(1168): n is a, c, g, or t
  misc_feature (1170)..(1172): n is a, c, g, or t
  misc_feature (1176)..(1176): n is a, c, g, or t
  misc_feature (1183)..(1184): n is a, c, g, or t
  misc_feature (1198)..(1199): n is a, c, g, or t
  misc_feature (1205)..(1207): n is a, c, g, or t
  misc_feature (1211)..(1219): n is a, c, g, or t
  misc_feature (1221)..(1221): n is a, c, g, or t
  misc_feature (1223)..(1226): n is a, c, g, or t
  misc_feature (1230)..(1235): n is a, c, g, or t
  misc_feature (1237)..(1239): n is a, c, g, or t
  misc_feature (1242)..(1246): n is a, c, g, or t
  misc_feature (1248)..(1248): n is a, c, g, or t
  misc_feature (1251)..(1254): n is a, c, g, or t
  misc_feature (1256)..(1258): n is a, c, g, or t
gnnnangctggnggcgtgcctaatacatgcaagtcgaacgaactctggtattgattggtgcttgcatcatgatttacatttgagtgagtggcgaactggtgagtaacacgtgggaaacctgcccaggngcgggggataacacctggaaacagatgctaataccgcataacaacttggaccgcatggtccgagtttgaaagatggcttcggctatcacttttggatggtcccgcggcgtattagctagatggtggggtaacggctcaccatggcaatgatacgtagccgacctgagagggtaatcggccacattgggactgagacacggcccaaactcctacgggaggcagcagtagggaatcttccacaatggacgaaagtctgatggagcaacgccgcgtgagtgaagaagggtttcggctcgtaaaactctgttgttaaagaagaacatatctgagagtaactgttcaggtattgacggtatttaaccagaaagccacggctaactacgtgccagcagccgcggtaatacgtaggtggcaagcgttgtccggatttattgggcgtaaagcgagcgcaggcggttttttaagtctgatgtgaaagccttcggctcaaccgaagaagtgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagtatgggtagcaaacaggattagataccctggtagtccataccgtaaacgatgaatgctaagtgttggagggtttccgcccttcagtgctgcagctaacgcattaagcattccgcctggggagtacggccgcaaggctgaaactcaaaggaattgacgggggcccgcacaagcggtggagcatgtggtttaattcgaagctacgcgaagaaccttaccaggtcttgacatactatgcaaatctaanagattagacgttcccttcggggacatggntacnggtggtgcatggttgtcgtcagctcgtgtcgtgagatgntgggntnaagtcccgcaacgancgcanccnnantatnagttgnccagcattaannnnnnnntctgnnanactnnnnnannnnnnnngnnngggntgacgtnnaatcatcatgcccnnatgacnnngctnnnnnnnnnanannnntggnnnnnnannnggnnnnntnccnnnnannn

SEQ ID NO: 2 - DNA, 1220 bp, Lactobacillus plantarum
  misc_feature (1)..(7): n is a, c, g, or t
  misc_feature (12)..(14): n is a, c, g, or t
  misc_feature (1026)..(1026): n is a, c, g, or t
  misc_feature (1052)..(1052): n is a, c, g, or t
  misc_feature (1061)..(1061): n is a, c, g, or t
  misc_feature (1072)..(1075): n is a, c, g, or t
  misc_feature (1082)..(1083): n is a, c, g, or t
  misc_feature (1085)..(1089): n is a, c, g, or t
  misc_feature (1091)..(1093): n is a, c, g, or t
  misc_feature (1096)..(1096): n is a, c, g, or t
  misc_feature (1112)..(1115): n is a, c, g, or t
  misc_feature (1120)..(1121): n is a, c, g, or t
  misc_feature (1130)..(1130): n is a, c, g, or t
  misc_feature (1132)..(1132): n is a, c, g, or t
  misc_feature (1137)..(1139): n is a, c, g, or t
  misc_feature (1141)..(1141): n is a, c, g, or t
  misc_feature (1145)..(1150): n is a, c, g, or t
  misc_feature (1156)..(1156): n is a, c, g, or t
  misc_feature (1158)..(1158): n is a, c, g, or t
  misc_feature (1160)..(1164): n is a, c, g, or t
  misc_feature (1166)..(1170): n is a, c, g, or t
  misc_feature (1172)..(1173): n is a, c, g, or t
  misc_feature (1176)..(1176): n is a, c, g, or t
  misc_feature (1179)..(1181): n is a, c, g, or t
  misc_feature (1184)..(1185): n is a, c, g, or t
  misc_feature (1187)..(1188): n is a, c, g, or t
  misc_feature (1191)..(1196): n is a, c, g, or t
  misc_feature (1198)..(1210): n is a, c, g, or t
  misc_feature (1212)..(1213): n is a, c, g, or t
  misc_feature (1216)..(1219): n is a, c, g, or t
nnnnnnntgtcnnnttaggcggctggttcctaaaaggttaccccaccgactttgggtgttacaaactctcatggtgtgacgggcggtgtgtacaaggcccgggaacgtattcaccgcggcatgctgatccgcgattactagcgattccgacttcatgtaggcgagttgcagcctacaatccgaactgagaatggctttaagagattagcttactctcgcgagttcgcaactcgttgtaccatccattgtagcacgtgtgtagcccaggtcataaggggcatgatgatttgacgtcatccccaccttcctccggtttgtcaccggcagtctcaccagagtgcccaacttaatgctggcaactgataataagggttgcgctcgttgcgggacttaacccaacatctcacgacacgagctgacgacaaccatgcaccacctgtatccatgtccccgaagggaacgtctaatctcttagatttgcatagtatgtcaagacctggtaaggttcttcgcgtagcttcgaattaaaccacatgctccaccgcttgtgcgggcccccgtcaattcctttgagtttcagccttgcggccgtactccccaggcggaatgcttaatgcgttagctgcagcactgaagggcggaaaccctccaacacttagcattcatcgtttacggtatggactaccagggtatctaatcctgtttgctacccatactttcgagcctcagcgtcagttacagaccagacagccgccttcgccactggtgttcttccatatatctacgcatttcaccgctacacatggagttccactgtcctcttctgcactcaagtttcccagtttccgatgcacttcttcggttgagccgaaagctttcacatcagacttaaaaaaccgcctgcgctcgctttacgcccaataaatcccggacaacgcttgccacctacgtattaccgcggctgctggcacgtagttagccgtggctttctggttaaataccgtcaatacctgaacagttactctnagatatgttcttctttaacaacaganttttacganccgaaaccccnnnnacttcanncnnnnncnnntcngacttcgtccatttgnnnnatccnnactgctgcntnccgtnnnantttnnnnnngttctnantnnnnngnnnnntnnccntcnnnagnngnnacnnnnnnannnnnnnnnnnnncnnttnnnnc

SEQ ID NO: 3 - DNA, 1259 bp, Lactobacillus plantarum
  misc_feature (2)..(5): n is a, c, g, or t
  misc_feature (7)..(8): n is a, c, g, or t
  misc_feature (12)..(13): n is a, c, g, or t
  misc_feature (129)..(129): n is a, c, g, or t
  misc_feature (1040)..(1040): n is a, c, g, or t
  misc_feature (1044)..(1044): n is a, c, g, or t
  misc_feature (1075)..(1075): n is a, c, g, or t
  misc_feature (1085)..(1085): n is a, c, g, or t
  misc_feature (1129)..(1129): n is a, c, g, or t
  misc_feature (1135)..(1136): n is a, c, g, or t
  misc_feature (1141)..(1142): n is a, c, g, or t
  misc_feature (1144)..(1144): n is a, c, g, or t
  misc_feature (1149)..(1149): n is a, c, g, or t
  misc_feature (1152)..(1153): n is a, c, g, or t
  misc_feature (1157)..(1157): n is a, c, g, or t
  misc_feature (1159)..(1159): n is a, c, g, or t
  misc_feature (1161)..(1163): n is a, c, g, or t
  misc_feature (1167)..(1173): n is a, c, g, or t
  misc_feature (1187)..(1188): n is a, c, g, or t
  misc_feature (1204)..(1205): n is a, c, g, or t
  misc_feature (1210)..(1210): n is a, c, g, or t
  misc_feature (1212)..(1215): n is a, c, g, or t
  misc_feature (1217)..(1223): n is a, c, g, or t
  misc_feature (1226)..(1228): n is a, c, g, or t
  misc_feature (1230)..(1235): n is a, c, g, or t
  misc_feature (1237)..(1241): n is a, c, g, or t
  misc_feature (1243)..(1246): n is a, c, g, or t
  misc_feature (1248)..(1253): n is a, c, g, or t
  misc_feature (1256)..(1256): n is a, c, g, or t
  misc_feature (1258)..(1258): n is a, c, g, or t
gnnnnannctgnnggcgtgcctaatacatgcaagtcgaacgaactctggtattgattggtgcttgcatcatgatttacatttgagtgagtggcgaactggtgagtaacacgtgggaaacctgcccaggngcgggggataacacctggaaacagatgctaataccgcataacaacttggaccgcatggtccgagtttgaaagatggcttcggctatcacttttggatggtcccgcggcgtattagctagatggtggggtaacggctcaccatggcaatgatacgtagccgacctgagagggtaatcggccacattgggactgagacacggcccaaactcctacgggaggcagcagtagggaatcttccacaatggacgaaagtctgatggagcaacgccgcgtgagtgaagaagggtttcggctcgtaaaactctgttgttaaagaagaacatatctgagagtaactgttcaggtattgacggtatttaaccagaaagccacggctaactacgtgccagcagccgcggtaatacgtaggtggcaagcgttgtccggatttattgggcgtaaagcgagcgcaggcggttttttaagtctgatgtgaaagccttcggctcaaccgaagaagtgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagtatgggtagcaaacaggattagataccctggtagtccataccgtaaacgatgaatgctaagtgttggagggtttccgcccttcagtgctgcagctaacgcattaagcattccgcctggggagtacggccgcaaggctgaaactcaaaggaattgacgggggcccgcacaagcggtggagcatgtggtttaattcgaagctacgcgaagaaccttaccaggtcttgacatactatgcaaatctaagagattagacgttcccttcggggacatggntacnggtggtgcatggttgtcgtcagctcgtgtcntgagatgttngggttaagtcccgcacgagcgcaccccttattatcagttgcccngcattnnttggnncntctgngannctgncngnnncaannnnnnntgggggatgacgtnnaatcatcatgcccctnngaacngnnnnannnnnnnctnnnannnnnngnnnnnannnnannnnnngcnana

SEQ ID NO: 4 - DNA, 1218 bp, Lactobacillus plantarum
  misc_feature (1)..(5): n is a, c, g, or t
  misc_feature (7)..(8): n is a, c, g, or t
  misc_feature (11)..(14): n is a, c, g, or t
  misc_feature (1026)..(1026): n is a, c, g, or t
  misc_feature (1030)..(1030): n is a, c, g, or t
  misc_feature (1046)..(1046): n is a, c, g, or t
  misc_feature (1048)..(1048): n is a, c, g, or t
  misc_feature (1052)..(1052): n is a, c, g, or t
  misc_feature (1068)..(1068): n is a, c, g, or t
  misc_feature (1083)..(1083): n is a, c, g, or t
  misc_feature (1086)..(1088): n is a, c, g, or t
  misc_feature (1090)..(1090): n is a, c, g, or t
  misc_feature (1094)..(1094): n is a, c, g, or t
  misc_feature (1097)..(1098): n is a, c, g, or t
  misc_feature (1100)..(1100): n is a, c, g, or t
  misc_feature (1113)..(1118): n is a, c, g, or t
  misc_feature (1120)..(1121): n is a, c, g, or t
  misc_feature (1124)..(1125): n is a, c, g, or t
  misc_feature (1133)..(1134): n is a, c, g, or t
  misc_feature (1142)..(1142): n is a, c, g, or t
  misc_feature (1145)..(1150): n is a, c, g, or t
  misc_feature (1157)..(1157): n is a, c, g, or t
  misc_feature (1161)..(1168): n is a, c, g, or t
  misc_feature (1170)..(1172): n is a, c, g, or t
  misc_feature (1176)..(1179): n is a, c, g, or t
  misc_feature (1181)..(1181): n is a, c, g, or t
  misc_feature (1189)..(1192): n is a, c, g, or t
  misc_feature (1194)..(1206): n is a, c, g, or t
  misc_feature (1208)..(1208): n is a, c, g, or t
  misc_feature (1212)..(1214): n is a, c, g, or t
  misc_feature (1216)..(1218): n is a, c, g, or t
nnnnnanntgnnnnttaggcggctggttcctaaaaggttaccccaccgactttgggtgttacaaactctcatggtgtgacgggcggtgtgtacaaggcccgggaacgtattcaccgcggcatgctgatccgcgattactagcgattccgacttcatgtaggcgagttgcagcctacaatccgaactgagaatggctttaagagattagcttactctcgcgagttcgcaactcgttgtaccatccattgtagcacgtgtgtagcccaggtcataaggggcatgatgatttgacgtcatccccaccttcctccggtttgtcaccggcagtctcaccagagtgcccaacttaatgctggcaactgataataagggttgcgctcgttgcgggacttaacccaacatctcacgacacgagctgacgacaaccatgcaccacctgtatccatgtccccgaagggaacgtctaatctcttagatttgcatagtatgtcaagacctggtaaggttcttcgcgtagcttcgaattaaaccacatgctccaccgcttgtgcgggcccccgtcaattcctttgagtttcagccttgcggccgtactccccaggcggaatgcttaatgcgttagctgcagcactgaagggcggaaaccctccaacacttagcattcatcgtttacggtatggactaccagggtatctaatcctgtttgctacccatactttcgagcctcagcgtcagttacagaccagacagccgccttcgccactggtgttcttccatatatctacgcatttcaccgctacacatggagttccactgtcctcttctgcactcaagtttcccagtttccgatgcacttcttcggttgagccgaaagctttcacatcagacttaaaaaaccgcctgcgctcgctttacgcccaataaatcccggacaacgcttgccacctacgtattaccgcggctgctggcacgtagttagccgtggctttctggttaaataccgtcaatacctgaacagttactctnaganatgttcttctttaacnanaganttttacgagcccgaancccttcttcactcangcnnntnctcntcnnantttcgtcattggnnnnnnannccnnactgctgnnccgtaganttnnnnnngtctcantccnnnnnnnnannncccnnnngncggctacnnnncnnnnnnnnnnnnnancccnnntnnn

SEQ ID NO: 5 - DNA, 1247 bp, Corynebacterium amycolatum
  misc_feature (2)..(7): n is a, c, g, or t
  misc_feature (10)..(12): n is a, c, g, or t
  misc_feature (843)..(843): n is a, c, g, or t
  misc_feature (912)..(912): n is a, c, g, or t
  misc_feature (925)..(925): n is a, c, g, or t
  misc_feature (965)..(965): n is a, c, g, or t
  misc_feature (995)..(995): n is a, c, g, or t
  misc_feature (997)..(997): n is a, c, g, or t
  misc_feature (1006)..(1007): n is a, c, g, or t
  misc_feature (1017)..(1018): n is a, c, g, or t
  misc_feature (1031)..(1031): n is a, c, g, or t
  misc_feature (1053)..(1053): n is a, c, g, or t
  misc_feature (1055)..(1055): n is a, c, g, or t
  misc_feature (1057)..(1058): n is a, c, g, or t
  misc_feature (1065)..(1065): n is a, c, g, or t
  misc_feature (1074)..(1074): n is a, c, g, or t
  misc_feature (1089)..(1089): n is a, c, g, or t
  misc_feature (1093)..(1094): n is a, c, g, or t
  misc_feature (1114)..(1114): n is a, c, g, or t
  misc_feature (1120)..(1122): n is a, c, g, or t
  misc_feature (1125)..(1126): n is a, c, g, or t
  misc_feature (1128)..(1133): n is a, c, g, or t
  misc_feature (1135)..(1139): n is a, c, g, or t
  misc_feature (1145)..(1147): n is a, c, g, or t
  misc_feature (1153)..(1154): n is a, c, g, or t
  misc_feature (1161)..(1161): n is a, c, g, or t
  misc_feature (1163)..(1164): n is a, c, g, or t
  misc_feature (1167)..(1167): n is a, c, g, or t
  misc_feature (1174)..(1177): n is a, c, g, or t
  misc_feature (1180)..(1180): n is a, c, g, or t
  misc_feature (1182)..(1182): n is a, c, g, or t
  misc_feature (1186)..(1186): n is a, c, g, or t
  misc_feature (1188)..(1191): n is a, c, g, or t
  misc_feature (1193)..(1195): n is a, c, g, or t
  misc_feature (1198)..(1200): n is a, c, g, or t
  misc_feature (1202)..(1208): n is a, c, g, or t
  misc_feature (1210)..(1212): n is a, c, g, or t
  misc_feature (1214)..(1226): n is a, c, g, or t
  misc_feature (1228)..(1228): n is a, c, g, or t
  misc_feature (1232)..(1237): n is a, c, g, or t
  misc_feature (1239)..(1247): n is a, c, g, or t
gnnnnnnctnnngcgtgcttaacacatgcaagtcgaacggtaaggctccagcttgctggggtacacgagtggcgaacgggtgagtaacacgtgggtgacctgccctgcacttcgggataagcctgggaaactgggtctaataccggataggactgcaccgtgagggtgtggtggaaagttttttcggtgtgggatgggcccgcggcctatcagcttgttggtggggtaatggcctaccaaggcggcgacgggtagccggcctgagagggtggacggccacattgggactgaaacacggcccaaactcctacgggaggcagcagtggggaatattgcacaatgggcggaagcctgatgcagcgacgccgcgtgggggatgacggccttcgggttgtaaactcctttcaccatcgacgaagggtttctgacggtagatggagaaaaagcaccggctaactacgtgccagcagccgcggtaatacgtagggtgcgagcgttgtccggaattactgggcgtaaagagctcgtaggtggtttgtcgcgtcgtctgtgaaattccggggcttaactccgggcgtgcaggcgatacgggcataacttgagtactgtaggggagactggaattcctggtgtagcggtgaaatgcgcagatatcaggaggaacaccggtggcgaaggcgggtctctgggcagtaactgacgctgaggagcgaaagcatggggagcgaacaggattagataccctggtagtccatgccgtaaacggtgggcgctaggtgtgggtttccttccacgggatccgtgccgtagctaacgcattaagcgccccgcctggggagtacngccgcaaggctaaaactcaaaggaattgacgggggcccgcacaagcggcggagcatgtggattaattcnatgcaacgcgaanaaccttacctgggcttgacatatacaagatcgcgccaganatggtgtttcccttgtggcttgtatacagntngtgcatggnngtcgtcagcnncgtgtcgtgagangttggggttaagtcccgcaacnancnncaacccnttgtcttangttgccagcacgtgntggnnggggactcgtaagaaactgnccgggnnnacnncnnnnnntnnnnngacgtnnntcaatnntgccctnanntcnggcttcnnnncangnctanannnngnnncannngnnnnnnnannncnnnnnnnnnnnnnantccnnnnnnannnnnnnnn

SEQ ID NO: 6 - DNA, 1237 bp, Corynebacterium amycolatum
  misc_feature (1)..(8): n is a, c, g, or t
  misc_feature (12)..(13): n is a, c, g, or t
  misc_feature (15)..(15): n is a, c, g, or t
  misc_feature (20)..(20): n is a, c, g, or t
  misc_feature (889)..(889): n is a, c, g, or t
  misc_feature (978)..(978): n is a, c, g, or t
  misc_feature (981)..(981): n is a, c, g, or t
  misc_feature (985)..(985): n is a, c, g, or t
  misc_feature (994)..(994): n is a, c, g, or t
  misc_feature (1006)..(1006): n is a, c, g, or t
  misc_feature (1011)..(1011): n is a, c, g, or t
  misc_feature (1019)..(1019): n is a, c, g, or t
  misc_feature (1039)..(1039): n is a, c, g, or t
  misc_feature (1045)..(1045): n is a, c, g, or t
  misc_feature (1047)..(1048): n is a, c, g, or t
  misc_feature (1056)..(1056): n is a, c, g, or t
  misc_feature (1061)..(1062): n is a, c, g, or t
  misc_feature (1064)..(1064): n is a, c, g, or t
  misc_feature (1070)..(1071): n is a, c, g, or t
  misc_feature (1080)..(1080): n is a, c, g, or t
  misc_feature (1083)..(1083): n is a, c, g, or t
  misc_feature (1088)..(1090): n is a, c, g, or t
  misc_feature (1092)..(1096): n is a, c, g, or t
  misc_feature (1103)..(1103): n is a, c, g, or t
  misc_feature (1107)..(1110): n is a, c, g, or t
  misc_feature (1112)..(1112): n is a, c, g, or t
  misc_feature (1114)..(1114): n is a, c, g, or t
  misc_feature (1122)..(1122): n is a, c, g, or t
  misc_feature (1132)..(1132): n is a, c, g, or t
  misc_feature (1135)..(1136): n is a, c, g, or t
  misc_feature (1140)..(1140): n is a, c, g, or t
  misc_feature (1142)..(1143): n is a, c, g, or t
  misc_feature (1145)..(1145): n is a, c, g, or t
  misc_feature (1147)..(1147): n is a, c, g, or t
  misc_feature (1151)..(1151): n is a, c, g, or t
  misc_feature (1153)..(1157): n is a, c, g, or t
  misc_feature (1161)..(1161): n is a, c, g, or t
  misc_feature (1163)..(1168): n is a, c, g, or t
  misc_feature (1171)..(1176): n is a, c, g, or t
  misc_feature (1178)..(1183): n is a, c, g, or t
  misc_feature (1185)..(1185): n is a, c, g, or t
  misc_feature (1187)..(1187): n is a, c, g, or t
  misc_feature (1190)..(1190): n is a, c, g, or t
  misc_feature (1193)..(1194): n is a, c, g, or t
  misc_feature (1196)..(1196): n is a, c, g, or t
  misc_feature (1200)..(1204): n is a, c, g, or t
  misc_feature (1207)..(1218): n is a, c, g, or t
  misc_feature (1220)..(1223): n is a, c, g, or t
  misc_feature (1225)..(1233): n is a, c, g, or t
  misc_feature (1236)..(1237): n is a, c, g, or t
nnnnnnnnccanncncttcnaagctccctaacgagttgaggccacgtggcttcgggtgttaccgactttcatgacgtgacgggcggtgtgtacaaggcccgggaacgtattcaccgcagcgttgctgatctgcgattactagcgactccgacttcacggggtcgagttgcagaccccgatccgaactgagaccggctttgcgggattagctcgccctcacggactggcaacccactgtaccgaccattgtagcatgtgtgaagccctggacataaggggcatgatgatttgacgtcatccccaccttcctccgagttaaccccggcagtttcttacgagtccccaccatcacgtgctggcaacataagacaagggttgcgctcgttgcgggacttaacccaacatctcacgacacgagctgacgacaaccatgcaccacctgtatacaagccacaagggaaacaccatctctggcgcgatcttgtatatgtcaagcccaggtaaggttcttcgcgttgcatcgaattaatccacatgctccgccgcttgtgcgggcccccgtcaattcctttgagttttagccttgcggccgtactccccaggcggggcgcttaatgcgttagctacggcacggatcccgtggaaggaaacccacacctagcgcccaccgtttacggcatggactaccagggtatctaatcctgttcgctccccatgctttcgctcctcagcgtcagttactgcccagagacccgccttcgccaccggtgttcctcctgatatctgcgcatttcaccgctacaccaggaattccagtctcccctacagtactcaagttatgcccgtatcgcctgcacgcccggagttaagccccggaatttcacagacnacgcgacaaaccacctacgagctctttacgcccagtaattccggacaacgctcgcaccctacgtattaccgcggctgctgggcacgtanttngccngtgctttcnttctccatctancgtcngaaacccnttcgtcgatggtgaaaggantttacnannccgaaagncagtnntncccccnngcggcgtcnctncatcnnntnnnnnccattgngcannnntncnccactgcngcctccgtanggnntctngnncngngtcntnnnnncaangnnnnnncgnnnnnncnnnnnnangnccnggnnanccgnnnnnccnnnnnnnnnnnnannnncnnnnnnnnncann

SEQ ID NO: 7 - DNA, 1264 bp, Micrococcus luteus
  misc_feature (1)..(6): n is a, c, g, or t
  misc_feature (9)..(9): n is a, c, g, or t
  misc_feature (13)..(13): n is a, c, g, or t
  misc_feature (26)..(26): n is a, c, g, or t
  misc_feature (965)..(965): n is a, c, g, or t
  misc_feature (1011)..(1011): n is a, c, g, or t
  misc_feature (1029)..(1029): n is a, c, g, or t
  misc_feature (1043)..(1043): n is a, c, g, or t
  misc_feature (1051)..(1051): n is a, c, g, or t
  misc_feature (1089)..(1089): n is a, c, g, or t
  misc_feature (1094)..(1094): n is a, c, g, or t
  misc_feature (1099)..(1099): n is a, c, g, or t
  misc_feature (1108)..(1109): n is a, c, g, or t
  misc_feature (1114)..(1114): n is a, c, g, or t
  misc_feature (1131)..(1135): n is a, c, g, or t
  misc_feature (1138)..(1138): n is a, c, g, or t
  misc_feature (1163)..(1163): n is a, c, g, or t
  misc_feature (1166)..(1167): n is a, c, g, or t
  misc_feature (1171)..(1172): n is a, c, g, or t
  misc_feature (1174)..(1177): n is a, c, g, or t
  misc_feature (1184)..(1184): n is a, c, g, or t
  misc_feature (1186)..(1186): n is a, c, g, or t
  misc_feature (1188)..(1191): n is a, c, g, or t
  misc_feature (1193)..(1193): n is a, c, g, or t
  misc_feature (1195)..(1195): n is a, c, g, or t
  misc_feature (1197)..(1197): n is a, c, g, or t
  misc_feature (1199)..(1200): n is a, c, g, or t
  misc_feature (1202)..(1207): n is a, c, g, or t
  misc_feature (1209)..(1220): n is a, c, g, or t
  misc_feature (1223)..(1225): n is a, c, g, or t
  misc_feature (1227)..(1231): n is a, c, g, or t
  misc_feature (1236)..(1242): n is a, c, g, or t
  misc_feature (1244)..(1244): n is a, c, g, or t
  misc_feature (1246)..(1256): n is a, c, g, or t
  misc_feature (1258)..(1258): n is a, c, g, or t
  misc_feature (1261)..(1264): n is a, c, g, or t
nnnnnnaanctgnggcgtgcttaacncatgcaagtcgaacgatgaagcccagcttgctgggtggattagtggcgaacgggtgagtaacacgtgagtaacctgcccttaactctgggataagcctgggaaactgggtctaataccggataggagcgccgaccgcatggtgggtgttggaaagatttatcggttttggatggactcgcggcctatcagcttgttggtgaggtaatggctcaccaaggcgacgacgggtagccggcctgagagggtgaccggccacactgggactgagacacggcccagactcctacgggaggcagcagtggggaatattgcacaatgggcgcaagcctgatgcagcgacgccgcgtgagggatgacggccttcgggttgtaaacctctttcagtagggaagaagcgaaagtgacggtacctgcagaagaagcaccggctaactacgtgccagcagccgcggtaatacgtagggtgcgagcgttatccggaattattgggcgtaaagagctcgtaggcggtttgtcgcgtctgtcgtgaaagtccggggcttaaccccggatctgcggtgggtacgggcagactagagtgcagtaggggagactggaattcctggtgtagcggtggaatgcgcagatatcaggaggaacaccgatggcgaaggcaggtctctgggctgtaactgacgctgaggagcgaaagcatggggagcgaacaggattagataccctggtagtccatgccgtaaacgttgggcactaggtgtggggaccattccacggtttccgcgccgcagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatgttctcgatcgccgtanagatacggtttcccctttggggcgggttcacaggtggtgcatggtngtcgtcagctcgtgtcgngagatgttgggttnaagtcccncaacgagcgcaaccctcgttccatgttgccagcacgtnatggnggggnctcatgggnnactgncggggtcaactcggagnnnnnagncgacgtcaatcatcatgccccttangtnntggnntnnnncatgctncnannnngntncnanngnnnnnnannnnnnnnnnnnggnnntnnnnnaaaannnnnnntnannnnnnnnnnngnggnnnn

SEQ ID NO: 8 - DNA, 1263 bp, Micrococcus luteus
  misc_feature (1)..(7): n is a, c, g, or t
  misc_feature (10)..(10): n is a, c, g, or t
  misc_feature (14)..(14): n is a, c, g, or t
  misc_feature (20)..(20): n is a, c, g, or t
  misc_feature (1051)..(1051): n is a, c, g, or t
  misc_feature (1065)..(1065): n is a, c, g, or t
  misc_feature (1082)..(1083): n is a, c, g, or t
  misc_feature (1103)..(1104): n is a, c, g, or t
  misc_feature (1119)..(1121): n is a, c, g, or t
  misc_feature (1125)..(1127): n is a, c, g, or t
  misc_feature (1133)..(1135): n is a, c, g, or t
  misc_feature (1137)..(1137): n is a, c, g, or t
  misc_feature (1142)..(1142): n is a, c, g, or t
  misc_feature (1144)..(1144): n is a, c, g, or t
  misc_feature (1148)..(1150): n is a, c, g, or t
  misc_feature (1154)..(1155): n is a, c, g, or t
  misc_feature (1157)..(1157): n is a, c, g, or t
  misc_feature (1164)..(1169): n is a, c, g, or t
  misc_feature (1172)..(1173): n is a, c, g, or t
  misc_feature (1175)..(1175): n is a, c, g, or t
  misc_feature (1181)..(1181): n is a, c, g, or t
  misc_feature (1184)..(1184): n is a, c, g, or t
  misc_feature (1187)..(1191): n is a, c, g, or t
  misc_feature (1193)..(1193): n is a, c, g, or t
  misc_feature (1195)..(1196): n is a, c, g, or t
  misc_feature (1200)..(1203): n is a, c, g, or t
  misc_feature (1206)..(1207): n is a, c, g, or t
  misc_feature (1211)..(1211): n is a, c, g, or t
  misc_feature (1213)..(1215): n is a, c, g, or t
  misc_feature (1217)..(1223): n is a, c, g, or t
  misc_feature (1225)..(1225): n is a, c, g, or t
  misc_feature (1227)..(1230): n is a, c, g, or t
  misc_feature (1232)..(1236): n is a, c, g, or t
  misc_feature (1238)..(1238): n is a, c, g, or t
  misc_feature (1240)..(1245): n is a, c, g, or t
  misc_feature (1248)..(1248): n is a, c, g, or t
  misc_feature (1251)..(1251): n is a, c, g, or t
  misc_feature (1254)..(1256): n is a, c, g, or t
  misc_feature (1258)..(1263): n is a, c, g, or t
nnnnnnnctntccnccttcnaggctccccccacaagggttaggccaccggcttcgggtgttaccgactttcgtgacttgacgggcggtgtgtacaaggcccgggaacgtattcaccgcagcgttgctgatctgcgattactagcgactccgacttcatggggtcgagttgcagaccccaatccgaactgagaccggctttttgggattagctccatctcacagtatcgcaacccattgtaccggccattgtagcatgcgtgaagcccaagacataaggggcatgatgatttgacgtcgtcctcaccttcctccgagttgaccccggcagtctcccatgagtccccaccattacgtgctggcaacatggaacgagggttgcgctcgttgcgggacttaacccaacatctcacgacacgagctgacgacaaccatgcaccacctgtgaacccgccccaaaggggaaaccgtatctctacggcgatcgagaacatgtcaagccttggtaaggttcttcgcgttgcatcgaattaatccgcatgctccgccgcttgtgcgggcccccgtcaattcctttgagttttagccttgcggccgtactccccaggcggggcacttaatgcgttagctgcggcgcggaaaccgtggaatggtccccacacctagtgcccaacgtttacggcatggactaccagggtatctaatcctgttcgctccccatgctttcgctcctcagcgtcagttacagcccagagacctgccttcgccatcggtgttcctcctgatatctgcgcattccaccgctacaccaggaattccagtctcccctactgcactctagtctgcccgtacccaccgcagatccggggttaagccccggactttcacgacagacgcgacaaaccgcctacgagctctttacgcccaataattccggataacgctcgcaccctacgtattaccgcggctgctggcacgtagttagcccggtgcttcttctgcaggtaccgtcactttcgcttcttccctactgaaagagtttacaacccgaanccgtcatccctcangcggcgtcgctgcatcnncttgcgcccattgtgcaatnntccccactgctgccnnngtannntctggnnngngtctnantccnnngtgnngncaccccnnnnnngcnntnccgtcntcnccnnnnnancnntacnnnncannagcntnnngnnnnnnncntnnnncnnnnncntnnnnnnccnaancannngnnnnnn

SEQ ID NO: 9 - DNA, 1275 bp, Lactococcus lactis
  misc_feature (1)..(3): n is a, c, g, or t
  misc_feature (5)..(7): n is a, c, g, or t
  misc_feature (11)..(11): n is a, c, g, or t
  misc_feature (20)..(20): n is a, c, g, or t
  misc_feature (1083)..(1083): n is a, c, g, or t
  misc_feature (1108)..(1110): n is a, c, g, or t
  misc_feature (1114)..(1114): n is a, c, g, or t
  misc_feature (1118)..(1118): n is a, c, g, or t
  misc_feature (1120)..(1120): n is a, c, g, or t
  misc_feature (1141)..(1144): n is a, c, g, or t
  misc_feature (1150)..(1150): n is a, c, g, or t
  misc_feature (1153)..(1153): n is a, c, g, or t
  misc_feature (1158)..(1158): n is a, c, g, or t
  misc_feature (1160)..(1160): n is a, c, g, or t
  misc_feature (1164)..(1164): n is a, c, g, or t
  misc_feature (1166)..(1166): n is a, c, g, or t
  misc_feature (1168)..(1172): n is a, c, g, or t
  misc_feature (1175)..(1175): n is a, c, g, or t
  misc_feature (1181)..(1181): n is a, c, g, or t
  misc_feature (1183)..(1183): n is a, c, g, or t
  misc_feature (1188)..(1193): n is a, c, g, or t
  misc_feature (1195)..(1197): n is a, c, g, or t
  misc_feature (1201)..(1207): n is a, c, g, or t
  misc_feature (1209)..(1218): n is a, c, g, or t
  misc_feature (1221)..(1222): n is a, c, g, or t
  misc_feature (1224)..(1224): n is a, c, g, or t
  misc_feature (1229)..(1229): n is a, c, g, or t
  misc_feature (1231)..(1234): n is a, c, g, or t
  misc_feature (1238)..(1239): n is a, c, g, or t
  misc_feature (1241)..(1241): n is a, c, g, or t
  misc_feature (1243)..(1244): n is a, c, g, or t
  misc_feature (1251)..(1258): n is a, c, g, or t
  misc_feature (1260)..(1261): n is a, c, g, or t
  misc_feature (1265)..(1266): n is a, c, g, or t
  misc_feature (1268)..(1274): n is a, c, g, or t
nnntnnnggtnttaccttangaagcgccctccttgcggttaggcaacctacttcgggtactcccaactcccgtggtgtgacgggcggtgtgtacaaggcccgggaacgtattcaccgcggcgtgctgatccgcgattactagcgattccgacttcatgtaggcgagttgcagcctacaatccgaactgagaatggttttaagagattagctaaacatcactgtctcgcgactcgttgtaccatccattgtagcacgtgtgtagcccaggtcataaggggcatgatgatttgacgtcatccccaccttcctccggtttatcaccggcagtctcgttagagtgcccaacttaatgatggcaactaacaataggggttgcgctcgttgcgggacttaacccaacatctcacgacacgagctgacgacaaccatgcaccacctgtatcccgtgtcccgaaggaacttcctatctctaggaatagcacgagtatgtcaagacctggtaaggttcttcgcgttgcttcgaattaaaccacatgctccaccgcttgtgcgggcccccgtcaattcctttgagtttcaaccttgcggtcgtactccccaggcggagtgcttattgcgttagctgcgatacagagaacttatagctccctacatctagcactcatcgtttacggcgtggactaccagggtatctaatcctgtttgctccccacgctttcgagcctcagtgtcagttacaggccagagagccgctttcgccaccggtgttcctccatatatctacgcatttcaccgctacacatggaattccactctcctctcctgcactcaagtctaccagtttccaatgcatacaatggttgagccactgccttttacaccagacttaataaaccacctgcgctcgctttacgcccaataaatcccggacaacgctcgggacctacgtattaccgcggctgctggcacgtagttagccgtccctttctgggtagttaccgtcacttgatgagctttccactctcaccaacgttcttctctaccaacagagttttacgatccgaaaacccttcttcactcangcgcgttgctcggtcagactttcgnnnttgncganantccctactgctgcctcccgtnnnnttgggncgngtctnantccnangnnnnnatnaccccntntcagnnnnnntnnntcannnnnnngnnnnnnnnnnctnncnactancnnnncaanncngnncatcttnnnnnnnngnnaatnngnnnnnnnc

SEQ ID NO: 10 - DNA, 1262 bp, Lactococcus lactis
  misc_feature (2)..(4): n is a, c, g, or t
  misc_feature (6)..(7): n is a, c, g, or t
  misc_feature (10)..(12): n is a, c, g, or t
  misc_feature (963)..(963): n is a, c, g, or t
  misc_feature (1067)..(1067): n is a, c, g, or t
  misc_feature (1080)..(1080): n is a, c, g, or t
  misc_feature (1085)..(1086): n is a, c, g, or t
  misc_feature (1098)..(1098): n is a, c, g, or t
  misc_feature (1112)..(1112): n is a, c, g, or t
  misc_feature (1115)..(1115): n is a, c, g, or t
  misc_feature (1123)..(1123): n is a, c, g, or t
  misc_feature (1135)..(1136): n is a, c, g, or t
  misc_feature (1142)..(1143): n is a, c, g, or t
  misc_feature (1146)..(1146): n is a, c, g, or t
  misc_feature (1150)..(1150): n is a, c, g, or t
  misc_feature (1152)..(1158): n is a, c, g, or t
  misc_feature (1164)..(1164): n is a, c, g, or t
  misc_feature (1174)..(1174): n is a, c, g, or t
  misc_feature (1177)..(1177): n is a, c, g, or t
  misc_feature (1188)..(1188): n is a, c, g, or t
  misc_feature (1192)..(1192): n is a, c, g, or t
  misc_feature (1194)..(1194): n is a, c, g, or t
  misc_feature (1196)..(1197): n is a, c, g, or t
  misc_feature (1199)..(1201): n is a, c, g, or t
  misc_feature (1204)..(1205): n is a, c, g, or t
  misc_feature (1208)..(1211): n is a, c, g, or t
  misc_feature (1216)..(1221): n is a, c, g, or t
  misc_feature (1224)..(1225): n is a, c, g, or t
  misc_feature (1227)..(1235): n is a, c, g, or t
  misc_feature (1237)..(1241): n is a, c, g, or t
  misc_feature (1245)..(1245): n is a, c, g, or t
  misc_feature (1248)..(1249): n is a, c, g, or t
  misc_feature (1253)..(1253): n is a, c, g, or t
  misc_feature (1255)..(1255): n is a, c, g, or t
  misc_feature (1261)..(1262): n is a, c, g, or t
gnnnannctnnngcgtgcctaatacatgcaagttgagcgctgaaggttggtacttgtaccgactggatgagcagcgaacgggtgagtaacgcgtggggaatctgcctttgagcgggggacaacatttggaaacgaatgctaataccgcataaaaactttaaacacaagttttaagtttgaaagatgcaattgcatcactcaaagatgatcccgcgttgtattagctagttggtgaggtaaaggctcaccaaggcgatgatacatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttggtagagaagaacgttggtgagagtggaaagctcatcaagtgacggtaactacccagaaagggacggctaactacgtgccagcagccgcggtaatacgtaggtcccgagcgttgtccggatttattgggcgtaaagcgagcgcaggtggtttattaagtctggtgtaaaaggcagtggctcaaccattgtatgcattggaaactggtagacttgagtgcaggagaggagagtggaattccatgtgtagcggtgaaatgcgtagatatatggaggaacaccggtggcgaaagcggctctctggcctgtaactgacactgaggctcgaaagcgtggggagcaaacaggattagataccctggtagtccacgccgtaaacgatgagtgctagatgtagggagctataagttctctgtatcgcagctaacgcaataagcactccgcctggggagtacgaccgcaaggttgaaactcaaaggaattgacgggggcccgcacaagcggtggagcatgtggtttaattcgaagcaacgcgaagaaccttaccnggtcttgacatactcgtgctattcctagagataggaagttccttcgggacacgggatacaggtggtgcatggttgtcgtcagctcgtgtcgtgagatgttgggnttaagtcccgcancgagnncaacccctattngttagttgccatcnttnagttgggncactctaacgannctgccnnganaaancnnnnnnntggggnatgacgtcantcntcatgcccctnatgnancnngnnnacnnacnnnnctacnnnnnnggnncnnnnnnnnngnnnnngtgnatnntttnancctaann

SEQ ID NO: 11 - DNA, 1263 bp, Lactococcus lactis
  misc_feature (1)..(6): n is a, c, g, or t
  misc_feature (10)..(14): n is a, c, g, or t
  misc_feature (18)..(18): n is a, c, g, or t
  misc_feature (1030)..(1030): n is a, c, g, or t
  misc_feature (1045)..(1045): n is a, c, g, or t
  misc_feature (1049)..(1049): n is a, c, g, or t
  misc_feature (1066)..(1066): n is a, c, g, or t
  misc_feature (1074)..(1074): n is a, c, g, or t
  misc_feature (1101)..(1101): n is a, c, g, or t
  misc_feature (1113)..(1113): n is a, c, g, or t
  misc_feature (1116)..(1116): n is a, c, g, or t
  misc_feature (1118)..(1119): n is a, c, g, or t
  misc_feature (1122)..(1122): n is a, c, g, or t
  misc_feature (1144)..(1145): n is a, c, g, or t
  misc_feature (1147)..(1148): n is a, c, g, or t
  misc_feature (1151)..(1151): n is a, c, g, or t
  misc_feature (1154)..(1156): n is a, c, g, or t
  misc_feature (1162)..(1162): n is a, c, g, or t
  misc_feature (1164)..(1164): n is a, c, g, or t
  misc_feature (1168)..(1169): n is a, c, g, or t
  misc_feature (1173)..(1175): n is a, c, g, or t
  misc_feature (1185)..(1190): n is a, c, g, or t
  misc_feature (1194)..(1198): n is a, c, g, or t
  misc_feature (1200)..(1201): n is a, c, g, or t
  misc_feature (1203)..(1209): n is a, c, g, or t
  misc_feature (1213)..(1215): n is a, c, g, or t
  misc_feature (1217)..(1226): n is a, c, g, or t
  misc_feature (1228)..(1228): n is a, c, g, or t
  misc_feature (1232)..(1232): n is a, c, g, or t
  misc_feature (1234)..(1236): n is a, c, g, or t
  misc_feature (1238)..(1238): n is a, c, g, or t
  misc_feature (1242)..(1243): n is a, c, g, or t
  misc_feature (1245)..(1249): n is a, c, g, or t
  misc_feature (1251)..(1255): n is a, c, g, or t
  misc_feature (1257)..(1259): n is a, c, g, or t
  misc_feature (1262)..(1262): n is a, c, g, or t
nnnnnncgtnnnnnttangagcgccctccttgcggttaggcaacctacttcgggtactcccaactcccgtggtgtgacgggcggtgtgtacaaggcccgggaacgtattcaccgcggcgtgctgatccgcgattactagcgattccgacttcatgtaggcgagttgcagcctacaatccgaactgagaatggttttaagagattagctaaacatcactgtctcgcgactcgttgtaccatccattgtagcacgtgtgtagcccaggtcataaggggcatgatgatttgacgtcatccccaccttcctccggtttatcaccggcagtctcgttagagtgcccaacttaatgatggcaactaacaataggggttgcgctcgttgcgggacttaacccaacatctcacgacacgagctgacgacaaccatgcaccacctgtatcccgtgtcccgaaggaacttcctatctctaggaatagcacgagtatgtcaagacctggtaaggttcttcgcgttgcttcgaattaaaccacatgctccaccgcttgtgcgggcccccgtcaattcctttgagtttcaaccttgcggtcgtactccccaggcggagtgcttattgcgttagctgcgatacagagaacttatagctccctacatctagcactcatcgtttacggcgtggactaccagggtatctaatcctgtttgctccccacgctttcgagcctcagtgtcagttacaggccagagagccgctttcgccaccggtgttcctccatatatctacgcatttcaccgctacacatggaattccactctcctctcctgcactcaagtctaccagtttccaatgcatacaatggttgagccactgccttttacaccagacttaataaaccacctgcgctcgctttacgcccaataaatccggacaacgctcgggacctacgtattaccgcggctgctggcacgtagttagccgtccctttctgggtagttaccgtcacttgatgagctttccactctcaccaacgnttcttctctaccaanaganttttacgatccgaaaanccttcttnactcacgcggcgtttgctcggtcaganttcgtccattgnccnannttnccctactgctgcctcccgtagnntnnggnccnnngttctnantccnntgtnnncgatcacccnnnnnnaggnnnnncnntnnnnnnnatcnnncnnnnnnnnnntncacntnnncnaacnnannnnnannnnnannncgnc

SEQ ID NO: 12 - DNA, 1269 bp, Bacillus subtilis
  misc_feature (2)..(4): n is a, c, g, or t
  misc_feature (6)..(9): n is a, c, g, or t
  misc_feature (11)..(12): n is a, c, g, or t
  misc_feature (1038)..(1038): n is a, c, g, or t
  misc_feature (1057)..(1057): n is a, c, g, or t
  misc_feature (1084)..(1084): n is a, c, g, or t
  misc_feature (1086)..(1086): n is a, c, g, or t
  misc_feature (1103)..(1103): n is a, c, g, or t
  misc_feature (1115)..(1115): n is a, c, g, or t
  misc_feature (1117)..(1117): n is a, c, g, or t
  misc_feature (1124)..(1125): n is a, c, g, or t
  misc_feature (1128)..(1129): n is a, c, g, or t
  misc_feature (1132)..(1132): n is a, c, g, or t
  misc_feature (1134)..(1134): n is a, c, g, or t
  misc_feature (1143)..(1144): n is a, c, g, or t
  misc_feature (1147)..(1149): n is a, c, g, or t
  misc_feature (1152)..(1152): n is a, c, g, or t
  misc_feature (1154)..(1154): n is a, c, g, or t
  misc_feature (1159)..(1159): n is a, c, g, or t
  misc_feature (1161)..(1161): n is a, c, g, or t
  misc_feature (1165)..(1165): n is a, c, g, or t
  misc_feature (1170)..(1170): n is a, c, g, or t
  misc_feature (1175)..(1175): n is a, c, g, or t
  misc_feature (1180)..(1181): n is a, c, g, or t
  misc_feature (1187)..(1187): n is a, c, g, or t
  misc_feature (1189)..(1189): n is a, c, g, or t
  misc_feature (1194)..(1201): n is a, c, g, or t
  misc_feature (1205)..(1211): n is a, c, g, or t
  misc_feature (1216)..(1226): n is a, c, g, or t
  misc_feature (1228)..(1236): n is a, c, g, or t
  misc_feature (1238)..(1247): n is a, c, g, or t
  misc_feature (1249)..(1249): n is a, c, g, or t
  misc_feature (1251)..(1253): n is a, c, g, or t
  misc_feature (1257)..(1259): n is a, c, g, or t
  misc_feature (1263)..(1269): n is a, c, g, or t
gnnnannnngnngcgtgcctaatacatgcaagtcgagcggacagatgggagcttgctccctgatgttagcggcggacgggtgagtaacacgtgggtaacctgcctgtaagactgggataactccgggaaaccggggctaataccggatggttgtttgaaccgcatggttcaaacataaaaggtggcttcggctaccacttacagatggacccgcggcgcattagctagttggtgaggtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtaccgttcgaatagggcggtaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggnttgtcgtcagctcgtgtcntgagatgttgggttaagtcccgcaacnancgcaacccttgatctanttgccagcattnanttgggcnnctnntangngactgccgnnacnnncgnangaagntngggntgacntcaantcatnntgcccntnatgannnnnnnnaccnnnnnnntacannnnnnnnnnnannnnnnnnncnnnnnnnnnncnannntcannncccnnnnnnn

SEQ ID NO: 13 - DNA, 1281 bp, Bacillus subtilis
  misc_feature (1)..(9): n is a, c, g, or t
  misc_feature (14)..(16): n is a, c, g, or t
  misc_feature (1012)..(1013): n is a, c, g, or t
  misc_feature (1051)..(1051): n is a, c, g, or t
  misc_feature (1077)..(1077): n is a, c, g, or t
  misc_feature (1089)..(1089): n is a, c, g, or t
  misc_feature (1093)..(1093): n is a, c, g, or t
  misc_feature (1108)..(1108): n is a, c, g, or t
  misc_feature (1111)..(1111): n is a, c, g, or t
  misc_feature (1121)..(1122): n is a, c, g, or t
  misc_feature (1124)..(1124): n is a, c, g, or t
  misc_feature (1127)..(1127): n is a, c, g, or t
  misc_feature (1148)..(1148): n is a, c, g, or t
  misc_feature (1155)..(1156): n is a, c, g, or t
  misc_feature (1158)..(1158): n is a, c, g, or t
  misc_feature (1160)..(1161): n is a, c, g, or t
  misc_feature (1163)..(1163): n is a, c, g, or t
  misc_feature (1165)..(1165): n is a, c, g, or t
  misc_feature (1170)..(1170): n is a, c, g, or t
  misc_feature (1173)..(1176): n is a, c, g, or t
  misc_feature (1179)..(1179): n is a, c, g, or t
  misc_feature (1186)..(1187): n is a, c, g, or t
  misc_feature (1192)..(1193): n is a, c, g, or t
  misc_feature (1196)..(1196): n is a, c, g, or t
  misc_feature (1200)..(1200): n is a, c, g, or t
  misc_feature (1205)..(1208): n is a, c, g, or t
  misc_feature (1211)..(1212): n is a, c, g, or t
  misc_feature (1214)..(1215): n is a, c, g, or t
  misc_feature (1217)..(1222): n is a, c, g, or t
  misc_feature (1224)..(1224): n is a, c, g, or t
  misc_feature (1226)..(1233): n is a, c, g, or t
  misc_feature (1235)..(1235): n is a, c, g, or t
  misc_feature (1237)..(1237): n is a, c, g, or t
  misc_feature (1239)..(1239): n is a, c, g, or t
  misc_feature (1241)..(1241): n is a, c, g, or t
  misc_feature (1246)..(1246): n is a, c, g, or t
  misc_feature (1249)..(1249): n is a, c, g, or t
  misc_feature (1251)..(1254): n is a, c, g, or t
  misc_feature (1258)..(1262): n is a, c, g, or t
  misc_feature (1264)..(1265): n is a, c, g, or t
  misc_feature (1267)..(1273): n is a, c, g, or t
  misc_feature (1275)..(1281): n is a, c, g, or t
nnnnnnnnntgtcnnnttcggcggctggctcctaaaaggttacctcaccgacttcgggtgttacaaactctcgtggtgtgacgggcggtgtgtacaaggcccgggaacgtattcaccgcggcatgctgatccgcgattactagcgattccagcttcacgcagtcgagttgcagactgcgatccgaactgagaacagatttgtgggattggcttaacctcgcggtttcgctgccctttgttctgtccattgtagcacgtgtgtagcccaggtcataaggggcatgatgatttgacgtcatccccaccttcctccggtttgtcaccggcagtcaccttagagtgcccaactgaatgctggcaactaagatcaagggttgcgctcgttgcgggacttaacccaacatctcacgacacgagctgacgacaaccatgcaccacctgtcactctgcccccgaaggggacgtcctatctctaggattgtcagaggatgtcaagacctggtaaggttcttcgcgttgcttcgaattaaaccacatgctccaccgcttgtgcgggcccccgtcaattcctttgagtttcagtcttgcgaccgtactccccaggcggagtgcttaatgcgttagctgcagcactaaggggcggaaaccccctaacacttagcactcatcgtttacggcgtggactaccagggtatctaatcctgttcgctccccacgctttcgctcctcagcgtcagttacagaccagagagtcgccttcgccactggtgttcctccacatctctacgcatttcaccgctacacgtggaattccactctcctcttctgcactcaagttccccagtttccaatgaccctccccggttgagccgggggctttcacatcagacttaagaaaccgcctgcgagccctttacgcccaataattcccggacaacgcttgccacctacgtattacccgcggctgctggcacgtagttagcccgtggctttctggttaggtaccgtcaannacccgccctattcgaacggtacttgttcttccctaacnacagagctttacgatcccgaaaaccnttcatcactcangcgngttgctccgtcaganttngtccattgcnnanatncctactgctgcctccgtagantctgggnngngnntnantccantgnnnnatnacccctnntcagnngcntacncatcnnnnccnngnngnnnnnnancnnnnnnnnancntnanggccnccncnnnnccannnnngnnannnnnnngnnnnnnn

SEQ ID NO: 14 - DNA, 1261 bp, Bacillus endophyticus
  misc_feature (2)..(4): n is a, c, g, or t
  misc_feature (7)..(7): n is a, c, g, or t
  misc_feature (10)..(12): n is a, c, g, or t
  misc_feature (958)..(958): n is a, c, g, or t
  misc_feature (1005)..(1005): n is a, c, g, or t
  misc_feature (1022)..(1022): n is a, c, g, or t
  misc_feature (1028)..(1029): n is a, c, g, or t
  misc_feature (1039)..(1040): n is a, c, g, or t
  misc_feature (1063)..(1063): n is a, c, g, or t
  misc_feature (1081)..(1081): n is a, c, g, or t
  misc_feature (1098)..(1099): n is a, c, g, or t
  misc_feature (1104)..(1104): n is a, c, g, or t
  misc_feature (1116)..(1117): n is a, c, g, or t
  misc_feature (1120)..(1120): n is a, c, g, or t
  misc_feature (1124)..(1124): n is a, c, g, or t
  misc_feature (1131)..(1133): n is a, c, g, or t
  misc_feature (1139)..(1139): n is a, c, g, or t
  misc_feature (1142)..(1142): n is a, c, g, or t
  misc_feature (1145)..(1155): n is a, c, g, or t
  misc_feature (1182)..(1182): n is a, c, g, or t
  misc_feature (1187)..(1188): n is a, c, g, or t
  misc_feature (1190)..(1190): n is a, c, g, or t
  misc_feature (1194)..(1196): n is a, c, g, or t
  misc_feature (1198)..(1203): n is a, c, g, or t
  misc_feature (1207)..(1213): n is a, c, g, or t
  misc_feature (1215)..(1222): n is a, c, g, or t
  misc_feature (1228)..(1228): n is a, c, g, or t
  misc_feature (1230)..(1233): n is a, c, g, or t
  misc_feature (1236)..(1237): n is a, c, g, or t
  misc_feature (1241)..(1242): n is a, c, g, or t
  misc_feature (1244)..(1245): n is a, c, g, or t
  misc_feature (1249)..(1249): n is a, c, g, or t
  misc_feature (1251)..(1252): n is a, c, g, or t
  misc_feature (1254)..(1254): n is a, c, g, or t
  misc_feature (1256)..(1257): n is a, c, g, or t
  misc_feature (1259)..(1259): n is a, c, g, or t
  misc_feature (1261)..(1261): n is a, c, g, or t
gnnnaanctnnngcgtgcctaatacatgcaagtcgagcggagttttgaaaagcttgcttttcaaaacttagcggcggacgggtgagtaacacgtgggcaacctgcccttgagacggggataactccgggaaaccggagctaataccggataacacatatcttcgcatgaggatatgttagaaggtggcttttagctaccactcaaggatgggcccgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtatctgttgaataagcaggtaccttgacggtacctaaccagaaagccacggctaactacgtgccagcagccgcggtaatacgtaggtggcaagcgttgtccggaattattgggcgtaaagcgcgcgcaggcggttccttaagtctgatgtgaaagcccacggctcaaccgtggagggtcattggaaactggggaacttgagtgcagaagaggagagcggaattccacgtgtagcggtgaaatgcgtagagatgtggaggaacaccagtggcgaaggcggctctctggtctgtaactgacgctgaggcgcgaaagcgtggggagcgaacaggattagataccctggtagtccacgccgtaaacgatgagtgctaagtgttagagggtttccgccctttagtgctgcagcaaacgcattaagcactccgcctggggagtacggtcgcaagactgaaactcaaaggaattgacggggacccgcacaagcggtggagcatgtggtttaattcgaagcaacgcgaanaaccttaccaggtcttgacatcctctgctacttctagagatagaagntcccttcggggacagantgacannggtgcatggnngtcgtcagctcgtgtcgtgagangttgggttaagtcccgcnacgagcgcaacccttgnncttanttgccagcattnnttngggnactctannngactgncgngannnnnnnnnnntggggatgacgtcaatcatcatgcccnttatnnanctgnnnannnnnnctannnnnnngnnnnnnnnctgcanannnngcnnggtnnanncccnanntncnncncn

SEQ ID NO: 15 - DNA, 1216 bp, Bacillus endophyticus
  misc_feature (1)..(3): n is a, c, g, or t
  misc_feature (5)..(6): n is a, c, g, or t
  misc_feature (10)..(10): n is a, c, g, or t
  misc_feature (12)..(13): n is a, c, g, or t
  misc_feature (138)..(138): n is a, c, g, or t
  misc_feature (656)..(656): n is a, c, g, or t
  misc_feature (726)..(726): n is a, c, g, or t
  misc_feature (745)..(745): n is a, c, g, or t
  misc_feature (796)..(796): n is a, c, g, or t
  misc_feature (931)..(931): n is a, c, g, or t
  misc_feature (956)..(956): n is a, c, g, or t
  misc_feature (958)..(958): n is a, c, g, or t
  misc_feature (964)..(964): n is a, c, g, or t
  misc_feature (980)..(980): n is a, c, g, or t
  misc_feature (987)..(988): n is a, c, g, or t
  misc_feature (994)..(996): n is a, c, g, or t
  misc_feature (999)..(999): n is a, c, g, or t
  misc_feature (1011)..(1011): n is a, c, g, or t
  misc_feature (1033)..(1033): n is a, c, g, or t
  misc_feature (1038)..(1040): n is a, c, g, or t
  misc_feature (1042)..(1042): n is a, c, g, or t
  misc_feature (1055)..(1059): n is a, c, g, or t
  misc_feature (1062)..(1063): n is a, c, g, or t
  misc_feature (1066)..(1068): n is a, c, g, or t
  misc_feature (1071)..(1071): n is a, c, g, or t
  misc_feature (1074)..(1074): n is a, c, g, or t
  misc_feature (1081)..(1082): n is a, c, g, or t
  misc_feature (1084)..(1084): n is a, c, g, or t
  misc_feature (1094)..(1096): n is a, c, g, or t
  misc_feature (1098)..(1099): n is a, c, g, or t
  misc_feature (1104)..(1104): n is a, c, g, or t
  misc_feature (1113)..(1114): n is a, c, g, or t
  misc_feature (1118)..(1118): n is a, c, g, or t
  misc_feature (1120)..(1120): n is a, c, g, or t
  misc_feature (1123)..(1123): n is a, c, g, or t
  misc_feature (1125)..(1125): n is a, c, g, or t
  misc_feature (1127)..(1135): n is a, c, g, or t
  misc_feature (1137)..(1138): n is a, c, g, or t
  misc_feature (1140)..(1146): n is a, c, g, or t
  misc_feature (1149)..(1151): n is a, c, g, or t
  misc_feature (1154)..(1157): n is a, c, g, or t
  misc_feature (1164)..(1165): n is a, c, g, or t
  misc_feature (1172)..(1178): n is a, c, g, or t
  misc_feature (1180)..(1184): n is a, c, g, or t
  misc_feature (1186)..(1187): n is a, c, g, or t
  misc_feature (1189)..(1190): n is a, c, g, or t
  misc_feature (1192)..(1195): n is a, c, g, or t
  misc_feature (1199)..(1199): n is a, c, g, or t
  misc_feature (1201)..(1205): n is a, c, g, or t
  misc_feature (1208)..(1216): n is a, c, g, or t
nnnanntgtncnnttcgcggctggctccaaaaggttacctcaccgacttcgggtgttacaaactctcgtggtgtgacgggcggtgtgtacaaaacccgggaacgtattcaccgcggcatgctgatccgcgattactancgattccggcttcatgtaggcaagttgcagcctacaatccgaactgaaaatggttttatgggattggcttaacctcgcggttttgcagccctttgtaccatccattgtaccacgtgtgtaccccaggtcataaggggcatgatgatttgacgtcatccccaccttcctccggtttgtcaccggcagtcaccttaaagtgcccaactaaatgctggcaactaaaatcaagggttgcgctcgttgcgggacttaacccaacatctcacgacacgagctgacaacaaccatgcaccacctgtcactctgtccccaaagggaaccttctatctctaaaattaccaaaggatgtcaagacctggtaaggttcttcgcgttgcttcaaattaaaccacatgctccaccgcttgtgcgggtccccgtcaattcctttgagtttcagtcttgcaaccgtactccccaggcggagtgcttaatgcgtttgctgcagcactaaagggcggaaaccctctaacacttancactcatcgtttacggcgtggactaccagggtatctaatcctgttcgctccccacgctttcgcgcctcancgtcagttacagaccaaanagccgccttcgccactggtgttcctccacatctctacgcatttcaccgctncacgtggaattccgctctcctcttctgcactcaagttccccagtttccaatgaccctccacggttgagccgtgggctttcacatcagacttaaggaaccgcctgcgcgcgctttacgcccaataatttccggacnacgcttgccacctacgtattaccgngntgctgnacgtagttagccgtgntttctgnnaggtannncangtacctgcttantcaacagatacttgttcttccntacannnanctttacgatccgnnnnnatnnctnnncgngtnctcgtcnnantttcgtcatnnngnnatccnactgctgcnncgtngnaancngnnnnnnnnnanncnnnnnnnatnnnccnnnngtcgctnncatcgtnnnnnnnannnnncnncnntnnnnatgncnnnnnccnnnnnnnnn

SEQ ID NO: 16 - DNA, 1284 bp, Bacillus subtilis
  misc_feature (2)..(7): n is a, c, g, or t
  misc_feature (11)..(11): n is a, c, g, or t
  misc_feature (1038)..(1039): n is a, c, g, or t
  misc_feature (1069)..(1069): n is a, c, g, or t
  misc_feature (1085)..(1085): n is a, c, g, or t
  misc_feature (1092)..(1092): n is a, c, g, or t
  misc_feature (1099)..(1100): n is a, c, g, or t
  misc_feature (1120)..(1120): n is a, c, g, or t
  misc_feature (1128)..(1129): n is a, c, g, or t
  misc_feature (1147)..(1147): n is a, c, g, or t
  misc_feature (1150)..(1152): n is a, c, g, or t
  misc_feature (1154)..(1155): n is a, c, g, or t
  misc_feature (1159)..(1160): n is a, c, g, or t
  misc_feature (1162)..(1164): n is a, c, g, or t
  misc_feature (1179)..(1182): n is a, c, g, or t
  misc_feature (1187)..(1188): n is a, c, g, or t
  misc_feature (1196)..(1196): n is a, c, g, or t
  misc_feature (1198)..(1199): n is a, c, g, or t
  misc_feature (1203)..(1204): n is a, c, g, or t
  misc_feature (1207)..(1208): n is a, c, g, or t
  misc_feature (1210)..(1212): n is a, c, g, or t
  misc_feature (1214)..(1214): n is a, c, g, or t
  misc_feature (1216)..(1219): n is a, c, g, or t
  misc_feature (1222)..(1222): n is a, c, g, or t
  misc_feature (1224)..(1224): n is a, c, g, or t
  misc_feature (1229)..(1241): n is a, c, g, or t
  misc_feature (1244)..(1246): n is a, c, g, or t
  misc_feature (1250)..(1252): n is a, c, g, or t
  misc_feature (1255)..(1260): n is a, c, g, or t
  misc_feature (1263)..(1269): n is a, c, g, or t
  misc_feature (1271)..(1271): n is a, c, g, or t
  misc_feature (1276)..(1276): n is a, c, g, or t
  misc_feature (1278)..(1278): n is a, c, g, or t
  misc_feature (1281)..(1284): n is a, c, g, or t
gnnnnnnctgnggcgtgcctaatacatgcaagtcgagcggacagatgggagcttgctccctgatgttagcggcggacgggtgagtaacacgtgggtaacctgcctgtaagactgggataactccgggaaaccggggctaataccggatggttgtttgaaccgcatggttcaaacataaaaggtggcttcggctaccacttacagatggacccgcggcgcattagctagttggtgaggtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtaccgttcgaatagggcggtaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtcccctttcggggcagagtgacaggtggtgcatggnngtcgtcagctcgtgtcgtgagatgttgggnttaagttcccgcaacnagcgcancccttgnncttagtttgccagcattcantttgggcnnctctaaggtgactgccgntgnnnannccgnngnnntgggggaatgacgtnnnntcatnntgcccctnanngacnnggnntnnncncnnnnctncnatggnnnnnnnnnnnnnagnnnaaannngcnnnnnnaannnnnnntnccccncnaannnn

SEQ ID NO: 17 - DNA, 1275 bp, Bacillus subtilis
  misc_feature (1)..(8): n is a, c, g, or t
  misc_feature (11)..(11): n is a, c, g, or t
  misc_feature (13)..(15): n is a, c, g, or t
  misc_feature (1049)..(1049): n is a, c, g, or t
  misc_feature (1053)..(1053): n is a, c, g, or t
  misc_feature (1102)..(1102): n is a, c, g, or t
  misc_feature (1115)..(1115): n is a, c, g, or t
  misc_feature (1118)..(1118): n is a, c, g, or t
  misc_feature (1120)..(1120): n is a, c, g, or t
  misc_feature (1122)..(1122): n is a, c, g, or t
  misc_feature (1125)..(1125): n is a, c, g, or t
  misc_feature (1141)..(1144): n is a, c, g, or t
  misc_feature (1149)..(1151): n is a, c, g, or t
  misc_feature (1153)..(1153): n is a, c, g, or t
  misc_feature (1158)..(1161): n is a, c, g, or t
  misc_feature (1163)..(1163): n is a, c, g, or t
  misc_feature (1165)..(1165): n is a, c, g, or t
  misc_feature (1167)..(1169): n is a, c, g, or t
  misc_feature (1173)..(1173): n is a, c, g, or t
  misc_feature (1176)..(1178): n is a, c, g, or t
  misc_feature (1182)..(1185): n is a, c, g, or t
  misc_feature (1188)..(1191): n is a, c, g, or t
  misc_feature (1196)..(1196): n is a, c, g, or t
  misc_feature (1200)..(1202): n is a, c, g, or t
  misc_feature (1204)..(1205): n is a, c, g, or t
  misc_feature (1208)..(1213): n is a, c, g, or t
  misc_feature (1216)..(1217): n is a, c, g, or t
  misc_feature (1223)..(1232): n is a, c, g, or t
  misc_feature (1235)..(1238): n is a, c, g, or t
  misc_feature (1240)..(1240): n is a, c, g, or t
  misc_feature (1242)..(1243): n is a, c, g, or t
  misc_feature (1246)..(1253): n is a, c, g, or t
  misc_feature (1257)..(1263): n is a, c, g, or t
  misc_feature (1265)..(1267): n is a, c, g, or t
  misc_feature (1271)..(1275): n is a, c, g, or t
nnnnnnnntgncnnnttcggcggctggctcctaaaaggttacctcaccgacttcgggtgttacaaactctcgtggtgtgacgggcggtgtgtacaaggcccgggaacgtattcaccgcggcatgctgatccgcgattactagcgattccagcttcacgcagtcgagttgcagactgcgatccgaactgagaacagatttgtgggattggcttaacctcgcggtttcgctgccctttgttctgtccattgtagcacgtgtgtagcccaggtcataaggggcatgatgatttgacgtcatccccaccttcctccggtttgtcaccggcagtcaccttagagtgcccaactgaatgctggcaactaagatcaagggttgcgctcgttgcgggacttaacccaacatctcacgacacgagctgacgacaaccatgcaccacctgtcactctgcccccgaaggggacgtcctatctctaggattgtcagaggatgtcaagacctggtaaggttcttcgcgttgcttcgaattaaaccacatgctccaccgcttgtgcgggcccccgtcaattcctttgagtttcagtcttgcgaccgtactccccaggcggagtgcttaatgcgttagctgcagcactaaggggcggaaaccccctaacacttagcactcatcgtttacggcgtggactaccagggtatctaatcctgttcgctccccacgctttcgctcctcagcgtcagttacagaccagagagtcgccttcgccactggtgttcctccacatctctacgcatttcaccgctacacgtggaattccactctcctcttctgcactcaagttccccagtttccaatgaccctccccggttgagccgggggctttcacatcagacttaagaaaccgcctgcgagccctttacgcccaataattcccggacaacgcttgccacctacgtattaccgcggctgctggcacgtagttagccgtggctttctggttaggtaccgtcaaggtaccgccctattcgaacggtacttgttcttccctaacanaganctttacgatccgaaacccttcatcactcacgcgcgttgctccgtcagantttcgtccattgnganantnccnactgctgccctccgtnnnntctgnnngngtctnnnncnantnnncccnatnnncccnnnnagnnnnctacncatnnntnnccnnnnnngannccgttnnnnnnnnnntannnnangnnccnnnnnnnncatnnnnnnnannngtgnnnnn

SEQ ID NO: 18 - DNA, 1273 bp, Bacillus amyloliquefaciens
  misc_feature (2)..(9): n is a, c, g, or t
  misc_feature (11)..(12): n is a, c, g, or t
  misc_feature (14)..(15): n is a, c, g, or t
  misc_feature (794)..(794): n is a, c, g, or t
  misc_feature (1081)..(1082): n is a, c, g, or t
  misc_feature (1093)..(1093): n is a, c, g, or t
  misc_feature (1097)..(1098): n is a, c, g, or t
  misc_feature (1103)..(1103): n is a, c, g, or t
  misc_feature (1120)..(1120): n is a, c, g, or t
  misc_feature (1124)..(1124): n is a, c, g, or t
  misc_feature (1130)..(1130): n is a, c, g, or t
  misc_feature (1140)..(1140): n is a, c, g, or t
  misc_feature (1142)..(1142): n is a, c, g, or t
  misc_feature (1148)..(1149): n is a, c, g, or t
  misc_feature (1152)..(1152): n is a, c, g, or t
  misc_feature (1154)..(1154): n is a, c, g, or t
  misc_feature (1156)..(1157): n is a, c, g, or t
  misc_feature (1161)..(1161): n is a, c, g, or t
  misc_feature (1165)..(1165): n is a, c, g, or t
  misc_feature (1177)..(1178): n is a, c, g, or t
  misc_feature (1186)..(1190): n is a, c, g, or t
  misc_feature (1193)..(1195): n is a, c, g, or t
  misc_feature (1200)..(1202): n is a, c, g, or t
  misc_feature (1205)..(1207): n is a, c, g, or t
  misc_feature (1209)..(1211): n is a, c, g, or t
  misc_feature (1215)..(1216): n is a, c, g, or t
  misc_feature (1220)..(1220): n is a, c, g, or t
  misc_feature (1223)..(1234): n is a, c, g, or t
  misc_feature (1236)..(1236): n is a, c, g, or t
  misc_feature (1238)..(1245): n is a, c, g, or t
  misc_feature (1248)..(1249): n is a, c, g, or t
  misc_feature (1252)..(1258): n is a, c, g, or t
  misc_feature (1260)..(1266): n is a, c, g, or t
  misc_feature (1269)..(1273): n is a, c, g, or t
gnnnnnnnngnngnngtgcctaatacatgcaagtcgagcggacagatgggagcttgctccctgatgttagcggcggacgggtgagtaacacgtgggtaacctgcctgtaagactgggataactccgggaaaccggggctaataccggatggttgtttgaaccgcatggttcaaacataaaaggtggcttcggctaccacttacagatggacccgcggcgcattagctagttggtgaggtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtaccgttcgaatagggcggtaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtancgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcannagcgcaacccnttgnncttanttgccagcattcagttnggcnactctnaggtgactgncnggtgannaancngnngaanggtnggggatgacgtnnaaatcatnnnnnccnnnatgannnggnnnannnaccnngctncannnnnnnnnnnnangnnnnnnnnaannccnnnnnnngnnnnnnnccnnnnn

SEQ ID NO: 19 - DNA, 1272 bp, Bacillus amyloliquefaciens
  misc_feature (1)..(4): n is a, c, g, or t
  misc_feature (6)..(7): n is a, c, g, or t
  misc_feature (12)..(14): n is a, c, g, or t
  misc_feature (1075)..(1075): n is a, c, g, or t
  misc_feature (1084)..(1084): n is a, c, g, or t
  misc_feature (1088)..(1088): n is a, c, g, or t
  misc_feature (1096)..(1096): n is a, c, g, or t
  misc_feature (1101)..(1101): n is a, c, g, or t
  misc_feature (1118)..(1121): n is a, c, g, or t
  misc_feature (1123)..(1123): n is a, c, g, or t
  misc_feature (1127)..(1127): n is a, c, g, or t
  misc_feature (1136)..(1136): n is a, c, g, or t
  misc_feature (1153)..(1153): n is a, c, g, or t
  misc_feature (1155)..(1158): n is a, c, g, or t
  misc_feature (1165)..(1165): n is a, c, g, or t
  misc_feature (1172)..(1174): n is a, c, g, or t
  misc_feature (1176)..(1176): n is a, c, g, or t
  misc_feature (1186)..(1187): n is a, c, g, or t
  misc_feature (1190)..(1190): n is a, c, g, or t
  misc_feature (1192)..(1192): n is a, c, g, or t
  misc_feature (1197)..(1197): n is a, c, g, or t
  misc_feature (1205)..(1205): n is a, c, g, or t
  misc_feature (1207)..(1209): n is a, c, g, or t
  misc_feature (1212)..(1222): n is a, c, g, or t
  misc_feature (1224)..(1224): n is a, c, g, or t
  misc_feature (1226)..(1226): n is a, c, g, or t
  misc_feature (1228)..(1234): n is a, c, g, or t
  misc_feature (1236)..(1236): n is a, c, g, or t
  misc_feature (1238)..(1238): n is a, c, g, or t
  misc_feature (1242)..(1242): n is a, c, g, or t
  misc_feature (1244)..(1244): n is a, c, g, or t
  misc_feature (1246)..(1246): n is a, c, g, or t
  misc_feature (1250)..(1252): n is a, c, g, or t
  misc_feature (1254)..(1256): n is a, c, g, or t
  misc_feature (1260)..(1261): n is a, c, g, or t
  misc_feature (1263)..(1271): n is a, c, g, or t
nnnnanntgtcnnnttcggcggctggctcctaaaaggttacctcaccgacttcgggtgttacaaactctcgtggtgtgacgggcggtgtgtacaaggcccgggaacgtattcaccgcggcatgctgatccgcgattactagcgattccagcttcacgcagtcgagttgcagactgcgatccgaactgagaacagatttgtgggattggcttaacctcgcggtttcgctgccctttgttctgtccattgtagcacgtgtgtagcccaggtcataaggggcatgatgatttgacgtcatccccaccttcctccggtttgtcaccggcagtcaccttagagtgcccaactgaatgctggcaactaagatcaagggttgcgctcgttgcgggacttaacccaacatctcacgacacgagctgacgacaaccatgcaccacctgtcactctgcccccgaaggggacgtcctatctctaggattgtcagaggatgtcaagacctggtaaggttcttcgcgttgcttcgaattaaaccacatgctccaccgcttgtgcgggcccccgtcaattcctttgagtttcagtcttgcgaccgtactccccaggcggagtgcttaatgcgttagctgcagcactaaggggcggaaaccccctaacacttagcactcatcgtttacggcgtggactaccagggtatctaatcctgttcgctccccacgctttcgctcctcagcgtcagttacagaccagagagtcgccttcgccactggtgttcctccacatctctacgcatttcaccgctacacgtggaattccactctcctcttctgcactcaagttccccagtttccaatgaccctccccggttgagccgggggctttcacatcagacttaagaaaccgcctgcgagccctttacgcccaataattcccggacaacgcttgccacctacgtattaccgcggctgctggcacgtagttagccgtggctttctggttaggtaccgtcaaggtaccgccctattcgaacggtacttgttcttccctaacaacagagctttacgatcccgaaaaccttnatcactcangcgncgtttgcnccgtnagactttcgtccattgnnnnantccnactgctgcntcccgtaggagtctggncnnnngtctcantccagtnnngncgatcacccnnctnangtcgntacgcatncnnnccnnnnnnnnnnntnancnnnnnnnancntaangncncccnnngnnncccnntnnnnnnnnng

SEQ ID NO: 20 - DNA, 1235 bp, Corynebacterium minutissimum
  misc_feature (2)..(4): n is a, c, g, or t
  misc_feature (6)..(9): n is a, c, g, or t
  misc_feature (12)..(13): n is a, c, g, or t
  misc_feature (49)..(49): n is a, c, g, or t
  misc_feature (712)..(712): n is a, c, g, or t
  misc_feature (816)..(816): n is a, c, g, or t
  misc_feature (953)..(955): n is a, c, g, or t
  misc_feature (962)..(962): n is a, c, g, or t
  misc_feature (980)..(980): n is a, c, g, or t
  misc_feature (990)..(991): n is a, c, g, or t
  misc_feature (1014)..(1014): n is a, c, g, or t
  misc_feature (1032)..(1033): n is a, c, g, or t
  misc_feature (1045)..(1045): n is a, c, g, or t
  misc_feature (1053)..(1053): n is a, c, g, or t
  misc_feature (1058)..(1060): n is a, c, g, or t
  misc_feature (1073)..(1074): n is a, c, g, or t
  misc_feature (1077)..(1077): n is a, c, g, or t
  misc_feature (1080)..(1081): n is a, c, g, or t
  misc_feature (1093)..(1095): n is a, c, g, or t
  misc_feature (1098)..(1099): n is a, c, g, or t
  misc_feature (1102)..(1102): n is a, c, g, or t
  misc_feature (1106)..(1109): n is a, c, g, or t
  misc_feature (1114)..(1114): n is a, c, g, or t
  misc_feature (1116)..(1116): n is a, c, g, or t
  misc_feature (1118)..(1121): n is a, c, g, or t
  misc_feature (1126)..(1133): n is a, c, g, or t
  misc_feature (1135)..(1136): n is a, c, g, or t
  misc_feature (1146)..(1146): n is a, c, g, or t
  misc_feature (1149)..(1149): n is a, c, g, or t
  misc_feature (1155)..(1155): n is a, c, g, or t
  misc_feature (1159)..(1160): n is a, c, g, or t
  misc_feature (1162)..(1162): n is a, c, g, or t
  misc_feature (1164)..(1167): n is a, c, g, or t
  misc_feature (1169)..(1177): n is a, c, g, or t
  misc_feature (1182)..(1183): n is a, c, g, or t
  misc_feature (1185)..(1186): n is a, c, g, or t
  misc_feature (1188)..(1189): n is a, c, g, or t
  misc_feature (1191)..(1225): n is a, c, g, or t
  misc_feature (1228)..(1234): n is a, c, g, or t
gnnnannnnctnnggcgtgcttaacacatgcaagtcgaacggaaaggcnctcttgcttgcaaggggtactcgagtggcgaacgggtgagtaacacgtgggtgatctgccttgcactctgggataagcttgggaaactgggtctaataccggatatgaactgcctttagtgtggtggttggaaagttttttcggtgcaaaatgagctcgcggcctatcaccttgttggtggggtaatggcctaccaaggcgtcaacgggtagccggcctgaaagggtgtacggccacattgggactgaaatacggcccaaactcctacgggaggcagcagtggggaatattgcacaatgggcggaagcctgatgcagcgaccccgcgtgggggatgacggccttcgggttgtaaactcctttcaacagggacaaacctttttgtgacggtacctgtataaaaagcaccggctaactacttgccaccagccgcggtaatacttagggtgcgagcgttgtccggaattactgggcgtaaaaagctcgtaggtggtttgtcgcgtcgtctgtgaaattccggggcttaacttcgggcgtgcaggcaatacgggcataacttgagtgctgtaggggaaactggaattcctggtgtaccggtgaaatgcgcaaatatcaggaggaacaccgatggcgaaggcaggtctctgggcagttactgacgctnaggagcgaaagcatgggtagcaaacaggattagataccctggtagtccatgccgtaaacggtgggcgctaggtgtagggggcttccacgtcttctgtgccgtanctaacgcattaagcgccccgcctggggagtacggccgcaaggctaaaactcaaaggaattgacgggggcccgcacaagcggcggagcatgtggattaatttcgatgcaacgcgaagaacccttacctgggcttgacnnnacactanattgctgcagagatgtantgtcccttgnngttggtgtacaggtggtgcatgntgtcgtcagctcgtgtcnngagatgtgggtnaagtcccncaacnnngcgcaacccttgnnttnatnnttgcagcatttnnnggnnctnatgnnnnactgncngnnnnactcnnnnnnnngnntgacgtcaantcntcatgncccnnangnnnngnnnnnnnnntgctnnanngnngnnnnnnnnnnnnnnnnnnnnnnnnnnnnnnnnnnnaannnnnnnt

SEQ ID NO: 21 - DNA, 1244 bp, Corynebacterium minutissimum
  misc_feature (1)..(11): n is a, c, g, or t
  misc_feature (13)..(14): n is a, c, g, or t
  misc_feature (989)..(989): n is a, c, g, or t
  misc_feature (1029)..(1031): n is a, c, g, or t
  misc_feature (1040)..(1040): n is a, c, g, or t
  misc_feature (1046)..(1048): n is a, c, g, or t
  misc_feature (1063)..(1063): n is a, c, g, or t
  misc_feature (1067)..(1067): n is a, c, g, or t
  misc_feature (1078)..(1080): n is a, c, g, or t
  misc_feature (1085)..(1086): n is a, c, g, or t
  misc_feature (1094)..(1094): n is a, c, g, or t
  misc_feature (1098)..(1098): n is a, c, g, or t
  misc_feature (1105)..(1105): n is a, c, g, or t
  misc_feature (1112)..(1112): n is a, c, g, or t
  misc_feature (1129)..(1131): n is a, c, g, or t
  misc_feature (1134)..(1136): n is a, c, g, or t
  misc_feature (1140)..(1140): n is a, c, g, or t
  misc_feature (1143)..(1145): n is a, c, g, or t
  misc_feature (1148)..(1152): n is a, c, g, or t
  misc_feature (1162)..(1166): n is a, c, g, or t
  misc_feature (1170)..(1170): n is a, c, g, or t
  misc_feature (1175)..(1175): n is a, c, g, or t
  misc_feature (1181)..(1181): n is a, c, g, or t
  misc_feature (1183)..(1184): n is a, c, g, or t
  misc_feature (1188)..(1195): n is a, c, g, or t
  misc_feature (1197)..(1203): n is a, c, g, or t
  misc_feature (1208)..(1212): n is a, c, g, or t
  misc_feature (1214)..(1217): n is a, c, g, or t
  misc_feature (1220)..(1220): n is a, c, g, or t
  misc_feature (1222)..(1222): n is a, c, g, or t
  misc_feature (1224)..(1226): n is a, c, g, or t
  misc_feature (1228)..(1238): n is a, c, g, or t
  misc_feature (1240)..(1241): n is a, c, g, or t
  misc_feature (1243)..(1244): n is a, c, g, or t
nnnnnnnnnnncnnttcgaagctccctaacaagtttaggccactggcttcgggtgttaccaactttcatgacgtgacgggcggtgtgtacaaggcccgggaacgtattcaccgcagcgttgctgatctgcgattactagcgactccgacttcatggggtcgagttgcagaccccaatccgaactaaggccggctttcagcgattcgctccacctcacagtgtcgctgcgcgttgtaccgaccattgtagcatgtgtgaagccctggacataaggggcatgatgatttgacgtcatccccaccttcctccgagttaaccccggcagtctctcatgagtccccaaccaaatgctggcaacataagacaagggttgcgctcgttgcgggacttaacccaacatctcacgacacgagctgacgacaaccatgcaccacctgtacaccaaccacaagggacactacatctctgcagcaatctagtgtatgtcaagcccaggtaaggttcttcgcgttgcatcgaattaatccacatgctccgccgcttgtgcgggcccccgtcaattcctttgagttttagccttgcggccgtactccccaggcggggcgcttaatgcgttagctacggcacagaagacgtggaagccccctacacctagcgcccaccgtttacggcatggactaccagggtatctaatcctgtttgctacccatgctttcgctcctcagcgtcagtaactgcccagagacctgccttcgccatcggtgttcctcctgatatctgcgcatttcaccgctacaccaggaattccagtctcccctacagcactcaagttatgcccgtatcgcctgcacgcccgaagttaagccccggaatttcacagacgacgcgacaaaccacctacgagctctttacgcccagtaattcccggacaacgctcgcaccctacgtattaccgcggctgctggcacgtagttagcccggtgcttctnatacaggtaccgtcacaaaaagctttcgtccctgtcgaannnagtttacancccgannnccgtcatccccccangcgngtcgctgcatnnngcttnncccattgngcanattcccnactgctncctcccgtagagtctgnnngtnnntcantcnnntgnnnnntacacccctnnnnngccnggctnccgtcnanncctnnnnnnnncnnnnnnnccacnnnnntnnnngcngngnnncnnnnnnnnnnntnngnn

SEQ ID NO: 22 - DNA, 1250 bp, Bacillus endophyticus
  misc_feature (2)..(7): n is a, c, g, or t
  misc_feature (9)..(9): n is a, c, g, or t
  misc_feature (11)..(12): n is a, c, g, or t
  misc_feature (1020)..(1020): n is a, c, g, or t
  misc_feature (1025)..(1027): n is a, c, g, or t
  misc_feature (1061)..(1062): n is a, c, g, or t
  misc_feature (1094)..(1094): n is a, c, g, or t
  misc_feature (1098)..(1098): n is a, c, g, or t
  misc_feature (1102)..(1103): n is a, c, g, or t
  misc_feature (1118)..(1118): n is a, c, g, or t
  misc_feature (1120)..(1120): n is a, c, g, or t
  misc_feature (1123)..(1124): n is a, c, g, or t
  misc_feature (1126)..(1127): n is a, c, g, or t
  misc_feature (1129)..(1131): n is a, c, g, or t
  misc_feature (1133)..(1133): n is a, c, g, or t
  misc_feature (1139)..(1139): n is a, c, g, or t
  misc_feature (1142)..(1142): n is a, c, g, or t
  misc_feature (1148)..(1148): n is a, c, g, or t
  misc_feature (1151)..(1151): n is a, c, g, or t
  misc_feature (1153)..(1157): n is a, c, g, or t
  misc_feature (1159)..(1161): n is a, c, g, or t
  misc_feature (1171)..(1176): n is a, c, g, or t
  misc_feature (1180)..(1184): n is a, c, g, or t
  misc_feature (1187)..(1187): n is a, c, g, or t
  misc_feature (1192)..(1193): n is a, c, g, or t
  misc_feature (1196)..(1196): n is a, c, g, or t
  misc_feature (1203)..(1203): n is a, c, g, or t
  misc_feature (1209)..(1215): n is a, c, g, or t
  misc_feature (1218)..(1232): n is a, c, g, or t
  misc_feature (1235)..(1237): n is a, c, g, or t
  misc_feature (1239)..(1240): n is a, c, g, or t
  misc_feature (1243)..(1243): n is a, c, g, or t
  misc_feature (1247)..(1250): n is a, c, g, or t
gnnnnnncngnngcgtgcctaatacatgcaagtcgagcggacagatgggagcttgctccctgatgttagcggcggacgggtgagtaacacgtgggtaacctgcctgtaagactgggataactccgggaaaccggggctaataccggatggttgtttgaaccgcatggttcaaacataaaaggtggcttcggctaccacttacagatggacccgcggcgcattagctagttggtgaggtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtaccgttcgaatagggcggtaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagantgacnnnggtgcatgggttgtcgtcagctcgtgtcgtgagnngttgggttaagtcccgcaacgagcgcaacccnttgntctnnttgccagcatttcantnggnncnncnnngngactgncgngacaancgnannnnngnnngacgtcaatnnnnnngccnnnnnganctggnnaanacacgtnctacannnnnnnaannnnnnnnnnnnnnnccnnngnnggnttannnn

SEQ ID NO: 23 - DNA, 1270 bp, Bacillus endophyticus
  misc_feature (2)..(8): n is a, c, g, or t
  misc_feature (11)..(11): n is a, c, g, or t
  misc_feature (13)..(15): n is a, c, g, or t
  misc_feature (1084)..(1084): n is a, c, g, or t
  misc_feature (1101)..(1101): n is a, c, g, or t
  misc_feature (1110)..(1111): n is a, c, g, or t
  misc_feature (1117)..(1120): n is a, c, g, or t
  misc_feature (1122)..(1122): n is a, c, g, or t
  misc_feature (1127)..(1127): n is a, c, g, or t
  misc_feature (1138)..(1138): n is a, c, g, or t
  misc_feature (1143)..(1145): n is a, c, g, or t
  misc_feature (1152)..(1152): n is a, c, g, or t
  misc_feature (1156)..(1157): n is a, c, g, or t
  misc_feature (1163)..(1169): n is a, c, g, or t
  misc_feature (1171)..(1175): n is a, c, g, or t
  misc_feature (1178)..(1180): n is a, c, g, or t
  misc_feature (1184)..(1187): n is a, c, g, or t
  misc_feature (1190)..(1191): n is a, c, g, or t
  misc_feature (1193)..(1195): n is a, c, g, or t
  misc_feature (1197)..(1197): n is a, c, g, or t
  misc_feature (1201)..(1201): n is a, c, g, or t
  misc_feature (1203)..(1211): n is a, c, g, or t
  misc_feature (1214)..(1216): n is a, c, g, or t
  misc_feature (1218)..(1219): n is a, c, g, or t
  misc_feature (1222)..(1225): n is a, c, g, or t
  misc_feature (1227)..(1227): n is a, c, g, or t
  misc_feature (1230)..(1232): n is a, c, g, or t
  misc_feature (1235)..(1236): n is a, c, g, or t
  misc_feature (1239)..(1243): n is a, c, g, or t
  misc_feature (1246)..(1253): n is a, c, g, or t
  misc_feature (1255)..(1256): n is a, c, g, or t
  misc_feature (1261)..(1263): n is a, c, g, or t
  misc_feature (1265)..(1269): n is a, c, g, or t
cnnnnnnntgncnnnttcggcggctggctcctaaaaggttacctcaccgacttcgggtgttacaaactctcgtggtgtgacgggcggtgtgtacaaggcccgggaacgtattcaccgcggcatgctgatccgcgattactagcgattccagcttcacgcagtcgagttgcagactgcgatccgaactgagaacagatttgtgggattggcttaacctcgcggtttcgctgccctttgttctgtccattgtagcacgtgtgtagcccaggtcataaggggcatgatgatttgacgtcatccccaccttcctccggtttgtcaccggcagtcaccttagagtgcccaactgaatgctggcaactaagatcaagggttgcgctcgttgcgggacttaacccaacatctcacgacacgagctgacgacaaccatgcaccacctgtcactctgcccccgaaggggacgtcctatctctaggattgtcagaggatgtcaagacctggtaaggttcttcgcgttgcttcgaattaaaccacatgctccaccgcttgtgcgggcccccgtcaattcctttgagtttcagtcttgcgaccgtactccccaggcggagtgcttaatgcgttagctgcagcactaaggggcggaaaccccctaacacttagcactcatcgtttacggcgtggactaccagggtatctaatcctgttcgctccccacgctttcgctcctcagcgtcagttacagaccagagagtcgccttcgccactggtgttcctccacatctctacgcatttcaccgctacacgtggaattccactctcctcttctgcactcaagttccccagtttccaatgaccctccccggttgagccgggggctttcacatcagacttaagaaaccgcctgcgagccctttacgcccaataattcccggacaacgcttgccacctacgtattaccgcggctgctggcacgtagttagccgtggctttctggttaggtaccgtcaaggtaccgccctattcgaacggtacttgttcttccctaacaacagagctttacgatccgaaaaccttcatcactcangcggcgttgctccgtcngactttcgnnattgcnnnnantcccnactgctgcctnccgtnnngtctggnccgnntctcannnnnnngnnnnngannncccnnnnagnngnnncncatncnnnnnnnnngannntnnccnnnnantannntannggnnnnnggnnnnnnnngnnaagtnnnannnnna

SEQ ID NO: 24 - DNA, 1443 bp, Bacillus subtilis
  misc_feature (1)..(12): n is a, c, g, or t
  misc_feature (25)..(25): n is a, c, g, or t
  misc_feature (29)..(29): n is a, c, g, or t
  misc_feature (40)..(41): n is a, c, g, or t
  misc_feature (44)..(45): n is a, c, g, or t
  misc_feature (1426)..(1426): n is a, c, g, or t
nnnnnnnnnnnncgtgcctaatacntgcnagtcgagcggnnagnngggagcttgctccctgatgttagcggcggacgggtgagtaacacgtgggtaacctgcctgtaagactgggataactccgggaaaccggggctaataccggatggttgtttgaaccgcatggttcaaacataaaaggtggcttcggctaccacttacagatggacccgcggcgcattagctagttggtgaggtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtaccgttcgaatagggcggtaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaacnttttaggagccagccgc

SEQ ID NO: 25 - DNA, 1451 bp, Pediococcus acidilactici
  misc_feature (12)..(12): n is a, c, g, or t
  misc_feature (16)..(16): n is a, c, g, or t
  misc_feature (44)..(44): n is a, c, g, or t
  misc_feature (512)..(512): n is a, c, g, or t
  misc_feature (532)..(532): n is a, c, g, or t
  misc_feature (550)..(550): n is a, c, g, or t
  misc_feature (561)..(562): n is a, c, g, or t
  misc_feature (567)..(567): n is a, c, g, or t
  misc_feature (572)..(573): n is a, c, g, or t
  misc_feature (580)..(581): n is a, c, g, or t
  misc_feature (602)..(602): n is a, c, g, or t
  misc_feature (609)..(609): n is a, c, g, or t
  misc_feature (624)..(624): n is a, c, g, or t
  misc_feature (733)..(733): n is a, c, g, or t
  misc_feature (756)..(756): n is a, c, g, or t
gtgcctaatacntgcnagtcgaacgaacttccgttaattgattntgacgtgcttgcactgaatgagattttaacacgaagtgagtggcggacgggtgagtaacacgtgggtaacctgcccagaagcaggggataacacctggaaacagatgctaataccgtataacagagaaaaccgcctggttttcttttaaaagatggctctgctatcacttctggatggacccgcggcgcattagctagttggtgaggtaacggctcaccaaggcgatgatgcgtagccgacctgagagggtaatcggccacattgggactgagacacggcccagactcctacgggaggcagcagtagggaatcttccacaatggacgcaagtctgatggagcaacgccgcgtgagtgaagaagggtttcggctcgtaaagctctgttgttaaagaagaacgtgggtgagagtaactgttcacccagtgacggtatttaaccagaaagccacggctaactacgtgccancagccgcggtaatacgtagntggcaagcgttatccggnatttattgggnntaaancgagnncaggcgnncttttaagtctaatgtgaaanccttcgnctcaaccgaagaagngcattggaaactgggagacttgagtgcagaagaggacagtggaactccatgtgtagcggtgaaatgcgtagatatatggaagaacaccagtggcgaaggcggctgtctngtctgtaactgacgctgaggctngaaagcatgggtagcgaacaggattagataccctggtagtccatgccgtaaacgatgattactaagtgttggagggtttccgcccttcagtgctgcagctaacgcattaagtaatccgcctggggagtacgaccgcaaggttgaaactcaaaagaattgacgggggcccgcacaagcggtggagcatgtggtttaattcgaagctacgcgaagaaccttaccaggtcttgacatcttctgccaacctaagagattaggcgttcccttcggggacagaatgacaggtggtgcatggttgtcgtcagctcgtgtcgtgagatgttgggttaagtcccgcaacgagcgcaacccttattactagttgccagcattcagttgggcactctagtgagactgccggtgacaaaccggaggaaggtggggacgacgtcaaatcatcatgccccttatgacctgggctacacacgtgctacaatggatggtacaacgagttgcgaaaccgcgaggtttagctaatctcttaaaaccattctcagttcggactgtaggctgcaactcgcctacacgaagtcggaatcgctagtaatcgcggatcagcatgccgcggtgaatacgttcccgggccttgtacacaccgcccgtcacaccatgagagtttgtaacacccaaagccggtggggtaaccttttaggagc

SEQ ID NO: 26 - DNA, 1429 bp, Unknown
  Match to Bacillus sonorensis\Bacillus subtilis\Bacillus licheniformis\Bacillus haynesii
  misc_feature (2)..(11): n is a, c, g, or t
  misc_feature (14)..(14): n is a, c, g, or t
  misc_feature (26)..(27): n is a, c, g, or t
  misc_feature (31)..(31): n is a, c, g, or t
  misc_feature (42)..(43): n is a, c, g, or t
  misc_feature (46)..(47): n is a, c, g, or t
  misc_feature (64)..(64): n is a, c, g, or t
  misc_feature (68)..(68): n is a, c, g, or t
  misc_feature (70)..(70): n is a, c, g, or t
  misc_feature (193)..(193): n is a, c, g, or t
  misc_feature (205)..(206): n is a, c, g, or t
  misc_feature (215)..(215): n is a, c, g, or t
  misc_feature (220)..(220): n is a, c, g, or t
  misc_feature (226)..(226): n is a, c, g, or t
  misc_feature (230)..(230): n is a, c, g, or t
  misc_feature (260)..(260): n is a, c, g, or t
  misc_feature (265)..(265): n is a, c, g, or t
  misc_feature (268)..(268): n is a, c, g, or t
  misc_feature (294)..(294): n is a, c, g, or t
  misc_feature (305)..(305): n is a, c, g, or t
  misc_feature (333)..(333): n is a, c, g, or t
  misc_feature (353)..(353): n is a, c, g, or t
  misc_feature (362)..(362): n is a, c, g, or t
  misc_feature (373)..(373): n is a, c, g, or t
  misc_feature (376)..(376): n is a, c, g, or t
  misc_feature (381)..(381): n is a, c, g, or t
  misc_feature (387)..(387): n is a, c, g, or t
  misc_feature (434)..(435): n is a, c, g, or t
  misc_feature (437)..(437): n is a, c, g, or t
  misc_feature (503)..(504): n is a, c, g, or t
  misc_feature (612)..(612): n is a, c, g, or t
  misc_feature (633)..(633): n is a, c, g, or t
  misc_feature (712)..(712): n is a, c, g, or t
  misc_feature (717)..(717): n is a, c, g, or t
gnnnnnnnnnngcncgtgcctaatanntgcnagtcgagcggnnagnngggagcttgctccctgntgtnancggcggacgggtgagtaacacgtgggtaacctgcctgtaagactgggataactccgggaaaccggggctaataccggatgcttgattgaaccgcatggttcaattataaaaggtggcttttanctaccacttacnnatggacccncggcncattanctanttggtgaggtaacggctcaccaaggcaacnatgcntanccaacctgaaagggtgatcggccacnctgggactganacacggcccaaactcctacgggaggcancattagggaatcttccgcantggacaaangtctgacggancancgccncgtgantgatgaaggttttcggatcgtaaaactctgttgttagggaaaaacanntnccgttcgaatagggcggtaccttgacggtacctaaccaaaaagccacggctaactacttgccaccnnccgcggtaatacttaggtggcaagcgttgtccggaattattgggcgtaaagcgcgcgcaggcggtttcttaagtctgatgtgaaagcccccggctcaaccggggaggntcattggaaactggggaactngagtgcagaagaggagagtggaattccacgtgtagcggtgaaatgcgtagagatgtggaggaacaccagtggcgaaggngactntctggtctgtaactgacgctgaggcgcgaaagcgtggggagcgaacaggattagataccctggtagtccacgccgtaaacgatgagtgctaagtgttagagggtttccgccctttagtgctgcagcaaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaaccctagagatagggcttccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatgggcagaacaaagggcagcgaagccgcgaggctaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaacc

SEQ ID NO: 27 - DNA, 1453 bp, Unknown
  Match to Lactobacillus plantarum\ Lactobacilllus pentosus
  misc_feature (13)..(13): n is a, c, g, or t
  misc_feature (17)..(17): n is a, c, g, or t
  misc_feature (176)..(176): n is a, c, g, or t
  misc_feature (246)..(246): n is a, c, g, or t
  misc_feature (541)..(541): n is a, c, g, or t
  misc_feature (1450)..(1450): n is a, c, g, or t
cgtgcctaatacntgcnagtcgaacgaactctggtattgattggtgcttgcatcatgatttacatttgagtgagtggcgaactggtgagtaacacgtgggaaacctgcccagaagcgggggataacacctggaaacagatgctaataccgcataacaacttggaccgcatggtccnagtttgaaagatggcttcggctatcacttttggatggtcccgcggcgtattagctagatggtggggtaanggctcaccatggcaatgatacgtagccgacctgagagggtaatcggccacattgggactgagacacggcccaaactcctacgggaggcagcagtagggaatcttccacaatggacgaaagtctgatggagcaacgccgcgtgagtgaagaagggtttcggctcgtaaaactctgttgttaaagaagaacatatctgagagtaactgttcaggtattgacggtatttaaccagaaagccacggctaactacgtgccagcagccgcggtaatacgtaggtggcaagcgttgtccggnatttattgggcgtaaagcgagcgcaggcggttttttaagtctgatgtgaaagccttcggctcaaccgaagaagtgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagtatgggtagcaaacaggattagataccctggtagtccataccgtaaacgatgaatgctaagtgttggagggtttccgcccttcagtgctgcagctaacgcattaagcattccgcctggggagtacggccgcaaggctgaaactcaaaggaattgacgggggcccgcacaagcggtggagcatgtggtttaattcgaagctacgcgaagaaccttaccaggtcttgacatactatgcaaatctaagagattagacgttcccttcggggacatggatacaggtggtgcatggttgtcgtcagctcgtgtcgtgagatgttgggttaagtcccgcaacgagcgcaacccttattatcagttgccagcattaagttgggcactctggtgagactgccggtgacaaaccggaggaaggtggggatgacgtcaaatcatcatgccccttatgacctgggctacacacgtgctacaatggatggtacaacgagttgcgaactcgcgagagtaagctaatctcttaaagccattctcagttcggattgtaggctgcaactcgcctacatgaagtcggaatcgctagtaatcgcggatcagcatgccgcggtgaatacgttcccgggccttgtacacaccgcccgtcacaccatgagagtttgtaacacccaaagtcggtggggtaaccttttaggaaccagccgcntaa

SEQ ID NO: 28 - DNA, 1436 bp, Bacillus subtilis
cgtgcctaatacatgcaagtcgagcggacagatgggagcttgctccctgatgttagcggcggacgggtgagtaacacgtgggtaacctgcctgtaagactgggataactccgggaaaccggggctaataccggatggttgtttgaaccgcatggttcaaacataaaaggtggcttcggctaccacttacagatggacccgcggcgcattagctagttggtgaggtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtaccgttcgaatagggcggtaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaaccttttaggagccagccgccgaag

SEQ ID NO: 29 - DNA, 1436 bp, Bacillus subtilis
cgtgcctaatacatgcaagtcgagcggacagatgggagcttgctccctgatgttagcggcggacgggtgagtaacacgtgggtaacctgcctgtaagactgggataactccgggaaaccggggctaataccggatggttgtttgaaccgcatggttcaaacataaaaggtggcttcggctaccacttacagatggacccgcggcgcattagctagttggtgaggtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtaccgttcgaatagggcggtaccttgacggtacctaaccagaaagccacggctaactacgtgccagcagccgcggtaatacgtaggtggcaagcgttgtccgggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaaccttttaggagccagccgccgaa

SEQ ID NO: 30 - DNA, 1436 bp, Bacillus subtilis
cgtgcctaatacatgcaagtcgagcggacagatgggagcttgctccctgatgttagcggcggacgggtgagtaacacgtgggtaacctgcctgtaagactgggataactccgggaaaccggggctaataccggatggttgtttgaaccgcatggttcaaacataaaaggtggcttcggctaccacttacagatggacccgcggcgcattagctagttggtgaggtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtaccgttcgaatagggcggtaccttgacggtacctaaccagaaagccacggctaactacgtgccagcagccgcggtaatacgtaggtggcaagcgttgtccgggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagaataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaaccttttaggagccagccgccga

(19) United States
(12) Patent Application Publication
(10) Pub. No.: US 2022/0184153 A1
(43) Pub. Date: Jun. 16, 2022

(54) METHOD OF TREATING BOVINE MASTITIS

(71) Applicants: The State of Israel, Ministry of Agriculture&Rural Development, Agricltural Research Oganization(ARO, Rishon-LeZion (IL); The State of Israel, Ministry of Agriculture & Rural Development, Kimron Veterinary Institute, Beit-Dagan (IL)

(72) Inventors: Moshe SHEMESH, ModiIn (IL); Shlomo BLUM, Rishon LeZion (IL)

(73) Assignees: The State of Israel, Ministry of Agriculture&Rural Development, Agricltural Research Oganization(ARO, Rishon-LeZion (IL); The State of Israel, Ministry of Agriculture & Rural Development, Kimron Veterinary Institute, Beit-Dagan (IL)

(21) Appl. No.: 17/598,384

(22) PCT Filed: Mar. 24, 2020
(86) PCT No.: PCT/IL2020/050348

§ 371 Date: Sep. 27, 2021

Related U.S. Application Data
(60) Provisional application No. 62/823,010, filed on Mar. 25, 2019.

(51) Int. Cl. A61K 35/747; A61K 35/742; A61K 35/744; A61K 35/741; A61K 9/00; A61P 31/04

(52) U.S. Cl. CPC A61K 35/747; A61K 35/742; A61P 31/04; A61K 35/741; A61K 9/0017; A61K 35/744

(57) ABSTRACT

A method of treating or preventing mastitis in a bovine animal comprising administering to the animal a therapeutically effective amount of particular microbes thereby treating or preventing mastitis in the animal.

### RELATED APPLICATIONS

This application claims the benefit of priority of U.S. Provisional Patent Application No. 62/823,010 filed 25 Mar. 2019, the contents of which are incorporated herein by reference in their entirety.

### SEQUENCE LISTING STATEMENT

The ASCII file, entitled 82071SequenceListing.txt, created on 22 Mar. 2020, comprising 126,655 bytes, submitted concurrently with the filing of this application is incorporated herein by reference.

### FIELD AND BACKGROUND OF THE INVENTION

The present invention, in some embodiments thereof, relates to probiotic compositions for the treatment of bovine mastitis and, more particularly, but not exclusively, compositions comprising biofilm-generating bacteria for the treatment of bovine mastitis.

Bovine mastitis is one of the most serious problems of the dairy industry that impacts milk production, composition and quality. It is highly expensive to treat, and as such it is a priority for the industry to find novel and sustainable solutions for mastitis treatment. The most prevalent treatment against bovine mastitis to date is the usage of antibiotics, which can be problematic from a health and environmental viewpoint.

Bacteria are known to the the main cause of bovine mastitis. Examples of bacteria that are associated with bovine mastitis include E. coli, coagulase negative staphylococci (CNS) and Staphylococcus aureus. Some of the bacterial strains known to cause bovine mastitis can also cause food poisoning in downstream applications.

The outside skin of the dairy cows' udder is constantly in an environment that is susceptible for microbial infections and contaminations. The cows' teat cistern and streak canal serve as the gate to the inside of the cows' udder and therefore their microbiota are constantly challenged by external bacterial contamination, hence making them good candidates for valuable isolates. Several studies were performed on the teats' cistern and streak canal microbiota of dairy cows⁸, some of which in relation to bovine mastitis^{9,10}. Maria Carolina Espeche at el¹¹ performed a study focusing on lactic acid bacteria (LAB) from the cows' streak canal, identifying Streptococcus bovis and Weissella paramesenteroides as the main strain isolates. The use of isolated bacteria as agents against pathogen bacteria is desired acid canserve the foodindustry for this purpose¹².

Background art includes Espeche, M. C, et al Veterinary microbiology 135, 346-357 (2009).

### SUMMARY OF THE INVENTION

According to an aspect of the present invention there is provided a method of treating or preventing mastitis in a bovine animal comprising administering to the animal a therapeutically effective amount of at least one microbe selected from the group consisting of Bacillus subtilis, Bacillus sonorensis, Bacillus licheniformis, vacililus firmus, Bacillus megaterium, B. endophyticus, Bacillus safensis, Bacillus endophyticus, Bacillus amyloliquefaciens, Corynebacterium minutissimum, Corynebacterium amycolatum, Corynebacterium xerosis, Lactobacillus pentosus, Leuconostoc pseudomesenteroides, Micrococcus luteus, Rumineliibacillus suwonensis and Pediococcus acidilactici thereby treating or preventing mastitis in the animal.

According to embodiments of the present invention, the at least one microbe is selected from the group consisting of Bacillus subtilis, Bacillus sonorensis, Bacillus licheniformis, Bacilllus firmus, Bacillus megaterium, B. endophyticus, Bacillus safensis, Bacillus endophyticus, Bacillus amyloliquefaciens, Lactobacillus pentosus, Leuconostoc pseudomesenteroides, Micrococcus luteus, Rummeliibacillus suwonensis and Pediococcus acidilaci.

According to embodiments of the present invention, the at least one microbe is Bacillus subtilis.

According to an aspect of the present invention there is provided a method of treating or preventing mastitis in a bovine animal comprising topically applying a therapeutically effective amount of a composition comprising at least one probiotic microbe and a biofilm-producing bacteria, under conditions that said probiotic microbe is encapsulated by the biofilm producing bacteria, to an udder of the animal thereby treating or preventing mastitis in the animal.

According to embodiments of the present invention, the probiotic microbe is identified by being present on an udder of a bovine animal which has had no mastitis events in at least two milking cycles.

According to embodiments of the present invention, the biofilm-producing bacteria is identified by being present on an udder of a bovine animal which has had no mastitis events in at least two milking cycle.

According to embodiments of the present invention, the administering comprises topically administering.

According to embodiments of the present invention, the microbe is applied in the form of a teat dip, as a cream, as a wash, or in the form of a spray.

According to embodiments of the present invention, the composition is applied in the form of a teat dip, as a wash, or in the form of a spray.

According to embodiments of the present invention, the microbe is of the Bacillus genus.

According to embodiments of the present invention, the biofilm-producing bacteria is of the Bacillus genus.

According to embodiments of the present invention, the biofilm-producing bacteria is of a genus selected from the group consisting of C. amycolatum, L. reuteri and L. lactis.

According to embodiments of the present invention, the probiotic is selected from the group consisting of Corynebacterium minutissimum, Corynebacterium xerosis, Leuconostoc pseudomesenteroides, Micrococcus luteus, Lactobacillus plantarum, Enterococcus hirae and Rummellibacillus suwonensis.

According to embodiments of the present invention, the mastitis is caused by a pathogen selected from the group consisting of S. aureus, E. coli and Coagulase Negative Staphylococcus (CNSs).

According to embodiments of the present invention, the biofilm-producing bacteria encapsulates the probiotic bacteria prior to the applying.

According to embodiments of the present invention, the biofilm-producing bacteria encapsulates the probiotic bacteria following the applying.

According to embodiments of the present invention, the biofilm-producing bacteria encapsulates the probiotic bacteria by:

(a) in vitro co-culturing the probiotic bacteria with the biofilm-producing bacteria in a growth substrate under conditions that generate a biofilm which comprises the probiotic bacteria and biofilm-producing bacteria; and

(b) isolating the biofilm from the growth substrate.

According to embodiments of the present invention, the growth substrate comprises a growth medium.

According to embodiments of the present invention, the growth medium is selected from the group consisting of LB, LBGM, milk and MRS.

According to embodiments of the present invention, the biofilm-producing bacteria are of the bacillus genus and the probiotic bacteria are of the lactobacillales order, the growth substrate is LBGM, milk or MRS.

According to embodiments of the present invention, the growth substrate is MRS.

According to embodiments of the present invention, the conditions comprise a pH of about 6.5-8.

According to embodiments of the present invention, the conditions comprise a pH of 6.8-7.5.

According to embodiments of the present invention, the growth substrate comprises acetoin.

According to embodiments of the present invention, the method further comprises dehydrating the biofilm following the isolating.

According to embodiments of the present invention, the probiotic microbe is a bacteria.

According to embodiments of the present invention, the bacteria comprise no more than 50 bacterial species.

According to embodiments of the present invention, the biofilm-producing bacteria are a single species of biofilm-producing bacteria.

According to an aspect of the present invention there is provided a composition of matter comprising at least two species of bacteria of a genus selected from the group consisting of Bacillus subtilis, Bacillus sonorensis, Bacillus lichemformis, Bacilllus firmus, Bacillus megateriun, B. endophyticus, Bacillus endophyticus, Bacillus amyloliquefaciens, Corynebacterium minutissimum, Corynebacterium amycolatum, Corynebacterium xerosis, Lactococcus lactis, Leuconostoc pseudomesenteroides, Micrococcus luteus, Lactobacillus plantarum, Rummeliibacillus suwonensis and Lactobacillus reuteri, wherein the composition of matter is formulated for topical delivery.

According to embodiments of the present invention, the at least two species of bacteria are of a genus selected from the group consisting of Bacillus subtilis, Bacillus sonorensis, Bacillus licheniformis, Bacillus firmus, Bacillus megateriwn, B. endophyticus, Bacillus safensis, Bacillus endophyticus, Bacillus amyloliquefaciens, Lactobacillus pentosus, Lactococcus lactis, Leuconostoc pseudomesenteroides, Micrococcus luteus, Rummeliibacillus suwonensis, Lactobacillus plantarum and Pediococcus acidilactici.

According to embodiments of the present invention, the one of the at least two species is Bacillus subtilis.

According to embodiments of the present invention, the composition comprises no more than 50 species of bacteria.

According to embodiments of the present invention, the at least one of the at least two species produces a biofilm.

According to embodiments of the present invention, the biofilm encapsulates the second of the at least two species.

According to embodiments of the present invention, the composition of matter is formulated as teat dip, a wash, a spray or a cream.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a schematic illustration of the pipeline filtration process to isolate relevant teat cistern and streak canal bacterial candidates for future mastitis treatment.

FIGS. 2A-B. The bacterial isolates selection and filtration stages (A) mastitis long term resistance dairy cows somatic cell counts (SCC) showing cows with the highest potential to harbor bacteria population with anti-mastitis characteristics and (B) the phylogenetic tree of the MALDI Biotyper isolate identification.

FIGS. 3A-C. Antimicrobial indexes of relevant isolates against (A) general food pathogens, (B) Coagulase Negative Staphylococcus (CNSs) and (C) known bovine mastitis causes bacteria pathogens.

FIGS. 4A-B. L. plantarum inhibit the growth of various pathogens by secretion of volatile compounds. L. plantarum was plated on the MRS side of LB-MRS divided plates and incubated in aerobic conditions at 37° C. for 3 days. Then the LB side of the plate was inoculated with different pathogens: B. cereus, E. coli, P. aeruginosa, S. typhimurium, and S. aureus. (A) Representative image demonstrating the lethal effect of volatile compound secretion from L. plantarum on E. coli survival. (B) Graphs showing survival rates.

### DESCRIPTION OF SPECIFIC EMBODIMENTS OF THE INVENTION

The present invention, in some embodiments thereof, relates to probiotic compositions for the treatment of bovine mastitis and, more particularly, but not exclusively, compositions comprising biofilm-generating bacteria for the treatment of bovine mastitis.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Bovine mastitis (BM) is a costly disease in dairy cattle production. The prevention and treatment of mastitis is typically performed by applying antimicrobial products that negatively affect milk quality.

The present inventors now propose the use of probiotic microorganisms for the prevention and treatment of this disease.

The present inventors analyzed bacteria that were found in the udder microbiome of dairy cows which showed a long-term resistance to mastitis and propose to use these bacteria as a probiotic for both the treatment and prevention of this disease. Specifically, the present inventors found 57 potential strains, out of a 345 initial isolates library, that showed interesting characteristics related to antimicrobial activity, biofilm formation and antibiotic susceptibility.

In one embodiment, there is provided a method of treating or preventing mastitis in a bovine animal comprising administering to the animal a therapeutically effective amount of at least one microbe selected from the group consisting of Bacillus subtilis, Bacillus sonorensis, Bacillus licheniformis, Bacillus firmus, Bacillus megaterium, B. endophyticus, Bacillus safensis, Bacillus endophyticus, Bacillus amyloliquefaciens, Corynebacterium minutissimun, Corynebacterium amycolatum, Corynebacteriun xerosis, Lactobacillus pentosus, Leuconostoc pseudomesenteroides, Micrococcus luteus, Rummeliibacillus suwonensis and Pediococcus acidilactici, thereby treating or preventing mastitis in the animal.

In another embodiment, the microbe is selected from the group consisting of Bacillus subtilis, Bacillus sonorensis, Bacillus licheniformis, Bacillus firmus, Bacillus megaterium, B. endophyticus, Bacillus safensis, Bacillus endophyticus, Bacillus amyloliquefaciens, Lactobacillus pentosus, Leuconostoc pseudomesenteroides, Micrococcus luteus, Rummeliibacillus suwonensis and Pediococcus acidilactici.

In still another embodiment, the microbe is selected from the group consisting of Bacillus subtilis, Pediococcus acidilactici, Bacillus sonorensis and Lactobacillus pentosus.

In still another embodiment, the microbe is of the genus Bacillus subtilis.

Methods of confirming the identity of a bacterial genus are known in the art and include MALDI TOFF and 16S rRNA sequencing.

Exemplary bacteria that may be used for treatment of mastitis are those that are at least 96%, 97%, 98%, 99% or even 100% identical to the above described species.

Exemplary bacterial species are those comprising the 16S rRNA sequence being at least at least 96%, 97%, 98%, 99% or even 100% identical as the sequences set forth in SEQ ID NOs: 1-30.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

Bovine mastitis is an inflammatory reaction of the mammary gland associated with a response to the presence of infecting pathogens. Although the disease is obvious upon direct observation in its clinical form, sub-clinical mastitis is more prevalent than clinical mastitis, since it usually precedes the clinical form and is more difficult to detect, the disease may become a prolonged infection and more difficult to overcome. Furthermore, the sub-clinical form of mastitis involves the presence of colonies of microorganisms that can lead to cross contamination and infection in other animals within the herd.

In one embodiment, the bovine mastitis is a clinically manifest bovine mastitis.

In another embodiment, the bovine mastitis is a subclinical bovine mastitis.

The bovine that is treated according to the present invention is typically a ruminant animal (e.g., dairy cow, bison, water buffalo, antelope, pronghorn, nilgai).

According to a specific embodiment, the bovine is a dairy cow.

According to a particular embodiment, the bovine is a lactating bovine.

Particular breeds of dairy cows which may be treated (either prophylactically or actively) according to this aspect of the present invention include, but are not limited to Ayrshire, Brown Swiss, Australian commercial dairy cow, Dairy shorthorn, Holstein, Guernsey, Sahiwal, Illawarra, Jersey, Meuse-Rhine-Issel, Red Poll, Simmental, Australian Red breed, Australian Friesian Sahiwal and Australian milking zebu, or crosses thereof.

The probiotic microbes which are administered to the bovine animals are typically administered topically to the teat/udder area. Thus, the microbes may be administered in the form of a teat dip, as a cream, as a wash, or in the form of a spray.

The present inventors propose administering at least one, at least two, at least three or more species of bacteria.

According to a specific embodiment, at least one of the bacteria which is administered is a biofilm producing bacteria.

The term “biofilm” as used herein refers to a community of bacteria that are comprised (e.g. embedded or encapsulated) in a matrix of extracellular polymeric substances that they have produced. Typically, the bacteria when present in the biofilm exhibit an altered phenotype with respect to growth rate and gene transcription in comparison to freely floating planktonic bacteria. Examples of extracellular polymeric substances which may be present in the biofilm include exopolysaccharides (such as those synthesized by the products of the epsA-O operon) and amyloid fibers (such as those encoded by tapA-sigW-tasA operon). Thus, the matrix typically comprises extracellular DNA and protein, as well as carbohydrates.

It will be appreciated that the biofilm-producing bacteria may also be beneficial in treating/preventing mastitis.

Preferably, the biofilm-producing bacteria is non-pathogenic (i.e. do not cause physical harm to, or disease in) to a human being or to the bovine subject.

In one embodiment, the biofilm-producing bacteria belong to the genus Bacillus.

As used herein, the term “genus Bacillus” includes all members known to those of skill in the art that produce a biofilm, including but not limited to B. subtilis, B. licheniformis, B. lentus, B. brevis, B. stearothermophilus, B. alkalophilus, B. amyloliquefaciens, B. clausii, B. halodurans, B. megateriun, B. coagulans, B. circulans, B. lautus, and B. thuringiensis.

According to a particular embodiment, the bacillus genus includes Bacillus subtilis, Bacillus sonorensis, Bacillus licheniformis, Bacillus firmus, Bacillus megaterium, B. endophyticus, Bacillus safensis, Bacillus endophyticus, Bacillus amyloliquefaciens.

It is recognized that the genus Bacillus continues to undergo taxonomical reorganization. Thus, it is intended that the genus include species that have been reclassified, including but not limited to such organisms as B. stearothermophilus, which is now named “Geobacillus stearothermophilus.” The production of resistant endospores in the presence of oxygen is considered the defining feature of the genus Bacillus, although this characteristic also applies to the recently named Alicyclobacillus, Amphibacillus, Aneurinibacillus, Anoxybacillus, Brevibacillus, Filobacillus, Gracilibacillus, Halobacillus, Paenibacillus, Salibacillus, Thermobacillis, Ureibacillus, and Virgibacillus.

In one embodiment, the biofilm-producing bacteria are of the species B. subtilis.

Exemplary strains of B. subtilis contemplated by the present invention include, but are not limited to B. subtilis MS1577 and 127185/2 (MS302; dairy isolate) and NCIB3610.

Exemplary strains of B. paralicheniformis contemplated by the present invention include, but are not limited to B. paralicheniformis MS303, and B. paralicheniformis S127.

Exemplary strains of B. licheniformis contemplated by the present invention include, but are not limited to B. licheniformis MS310, and B. licheniformis MS307.

According to a particular embodiment, the biofilm-producing bacteria does not comprise the species B. cereus.

According to a particular embodiment, the bacillus biofilm-producing bacteria is Bacillus subtilis, Bacillus endophyticus, Bacillus safensis and/or Bacillus amyloliquefaciens

According to another embodiment, the biofilm-producing bacteria is of a genus selected from the group consisting of C. amycolatum, L. reuteri and L. lactis.

In one embodiment, the biofilm-producing bacteria are cultured prior to administration such that they produce a biofilm.

The biofilm producing bacteria may be co-cultured with the additional beneficial bacteria prior to administration.

In another embodiment, the biofilm-producing bacteria are co-cultured with the additional beneficial bacteria under conditions that enhance the encapsulation of the additional beneficial bacteria into the biofilm.

Examples of beneficial bacteria for the treatment of bovine mastitis include, but are not limited to Corynebacterium minutissimum, Corynebacterium xerosis, Leuconostoc pseudomesenteroides, Micrococcus luteus, Lactobacillus plantarum, Enterococcus hirae and Rummeliibacillus suwonensis.

Additional examples include those provided herein below: Streptococcus bovis CRL 1710, Streptococcus bovis CRL 1715, Streptococcus bovis CRL 1717, Streptococcus bovis CRL 1718, Streptococcus bovis CRL 1725, Streptococcus bovis CRL 1726, Streptococcus bovis CRL 1728, Streptococcus bovis CRL 1729, Streptococcus bovis CRL 1730, Streptococcus bovis CRL 1731, Streptococcus bovis CRL 1737, Streptococcus bovis CRL 1738, Streptococcus bovis CRL 1739, Streptococcus bovis CRL 1742, Streptococcus bovis CRL 1743, Streptococcus bovis CRL 1745, Lactobacillus brevis CRL 1711, Enterococcus hirae CRL 1712, Enterococcus hirae CRL 1732, Enterococcus mundtii CRL 1713, Enterococcus mundtii CPL 1656, Lactobacillus planatarum CRL 1716, Weissella paramesenteroides CRL 1719, Weissella paramesenteroides CRL 1721, Weissella paramesenteroides CRL 1723 Weissella paramesenteroides CRL 1733, Weissella paramesenteroides CRL 1740, Weissella paramesenteroides CRL 1741, Enterococcus saccharominimus CRL 1720, Enterococcus saccharolyticus CRL 1735, Lactobacillus perolens CRL 1724, Lactococcus lactis subsp lactis CRL 1655, Lactococcus lactis subsp lactis CRL 1736, Lactococcus lactis subsp lactis CRL 1744, Lactococcus lactis subsp lactis CRL 1746, Lactobacillus reuteri CRL 1734 and Enterococcus faecium CRL 1657.

In order to generate a co-culture, typically both the beneficial culture and the biofilm-generating culture are cultured separately to generate a starter culture. The medium and conditions of the starter culture are typically selected so as to optimize growth of each of the bacteria.

Contemplated started cultures include a dried starter culture, a dehydrated starter culture, a frozen starter culture, or a concentrated starter culture.

The starter culture is grown for at least two hours, 4 hours, 8 hours, 12 hours until a sufficient amount of bacteria are propagated.

The method of co-culturing the beneficial bacteria with the biofilm producing bacteria is selected such that it enables the proliferation of both types of microorganisms and, according to a particular embodiment, incorporation of both microorganisms into the biofilm.

In one embodiment, the co-culturing is carried out in (or on) a growth substrate that is typically used to culture the beneficial bacteria. The growth substrate may be a solid or a liquid medium. Preferably, the co-culture is shaken during the culturing.

Examples of growth substrates that can be used to culture bacteria include but are not limited to MRS medium, LB medium, TBS medium, yeast extract, soy peptone, casein peptone and meat peptone.

Further examples of media are listed in Table 1 herein below.

TABLE 1
Abiotrophia media - Recipe for medium appropriate for growth of Abiotrophia genus
Acetamide Medium - Recipe for Acetamide medium.
Acetobacter Medium - Recipe for medium appropriate for the growth of Acetobacter genus.
Actinoplanes Medium - Media used to grow certain Actinoplanes species
Agrobacterium Agar Recipe - Agar appropriate for growth of Agrobacterium genus
Alicyclobacillus Agar - Recipe for Alicyclobacillus Agar.
Alicyclobacillus Medium - Recipe for Alicyclobacillus Medium.
Allantoin mineral agar - Recipe for the preparation of Allantoin minimal agar.
Allantoin mineral medium - Recipe for the preparation of allantoin minimal medium.
Ashbya Full Medium - Recipe for the production of Ashbya full medium.
Azotobacter Agar - Agar appropriate for growth of Azobacter genus.
Bennett's Medium - media used for growth of some Actinoplanes species.
Bacillus agar - Agar used to grow some Bacillus species.
Bacillus broth - Agar used to grow some Bacillus species,
Bacillus schlegelii Medium - Medium appropriate for the growth of Bacillus schlegelii.
Bifidobacterium Medium - Recipe for Bifidobacterium medium.
Blue green algae agar - Recipe for blue green algae agar.
Blue green algae broth - Recipe for blue green algae broth.
Brain Heart Infusion Glucose Agar - Recipe for Brain Heart infusion Glucose Agar.
Caulobacter Agar - Recipe for Caulobacter Agar.
Caulobacter Medium - Recipe for Caulobacter Medium.
Cantharellus Agar Recipe - Recipe for Cantharellus agar,
CASO agar - Recipe for CASO agar.
Clostridium thermocellum Medium - Recipe for medium appropriate for growth of
Clostridium thermocellum
Corynebacterium agar - Recipe for Corynebacterium agar.
Creatinine Medium - Recipe for the production of creatinine medium.
Czapek Agar (CZA) - Recipe for Czapek Agar (CZA),
Desulfovibrio Medium - Recipe for Desulfbvibrio Medium.
Gluconobacter agar - Recipe for Gluconobacter agar.
Glucose Peptone Yeast Extract Agar (GPYA) - Recipe for Glucose Peptone Yeast
Extract Agar (GPYA).
Glucose Yeast Extract Agar - Recipe for Glucose Yeast Extract Agar.
Halobacterium agar - Recipe for the preparation of Halobacterium agar.
Halobacteria Medium - Recipe for Halobacteria Medium.
LB Agar - Recipe for the preparation of LB agar bacterial media.
LB broth - Recipe for the preparation of LB broth bacterial media.
LB broth (low salt) - Recipe for the preparation of low salt LB broth bacterial media.
Luminous Medium - Recipe for Luminous Medium.
M17 media - Recipe for the preparation M17 media.
M9 minimal media - Minimal salts bacterial media.
Mannitol agar - Recipe for mannitol agar,
Mannitol broth - Recipe for mannitol broth.
Marine agar - Recipe for marine agar. Used for the growth of several marine bacteria.
Marine broth - Recipe for marine broth. Used for the growth of several marine bacteria.
Methylarnine Salts Agar - Recipe for methylamine salts agar
Methylarnine Salts Medium - Recipe for methylarnine salts medium
Modified Chopped Meat Medium - Used for the growth of several aerobic bacteria.
MY medium - Maltose yeast extract bacterial growth medium,
N4 Mineral Medium - Recipe for the production of N4 mineral medium.
Nitrosomonas europaea medium - Recipe for the production of Nitrosomonas europaea medium.
Nutrient agar - Recipe for nutrient agar suitable for growth of many bacterial species.
Nutrient broth - Recipe for nutrient broth suitable for growth of many bacterial species,
MRS media - Recipe for MRS media, MRS media has been used for the recovery of lactic acid
bacteria (LAB) from various food products.
MS-Medium - Recipe for MS-medium.
N-Z amine agar with soluble starch and glucose - Agar used to grow some Actinomadura species
NZCYM - NZ amine, NaCl, bacto-yeast extract, casamino acids, and magnesium sulfate,
NZM - NZ amine, NaCl, and magnesium sulfate,
NZYM - NZ amine, NaCl, bacto-yeast extract, and magnesium sulfate.
Oatmeal agar - agar used to grow some Actinomadura species.
Oenococcus Medium - Recipe for the preparation of Oenococcus medium,
Osmophilic Agar - Recipe for Osmophilic Agar.
Osmophilic Medium - Recipe for Osmophilic Medium,
Phenol red lactose broth - turns yellow when lactose is fermented.
Potato-Canrot Medium - agar used to grow some Actinoplanes species.
Propionibacterium Agar Recipe - Agar appropriate for the growth of Propionibacterium.
Propionibacterium Medium Recipe - Medium appropriate for the growth of Propionibacterium.
PYS agar - agar used to grow some Actinomadura species.
R Medium - R Medium Recipe.
Rolled Oats Mineral Agar - Recipe for Roled Oats Minera Agar.
Saccharose agar - Recipe for the production of saccharose agar
Saccharose medium - Recipe for the production of saccharose medium
SOB media - Tryptone/yeast extract bacterial media.
SOC media - Tryptone/yeast extract bacterial media.
5% Sorbitol agar - Recipe for the production of 5% sorbitol agar,
5% Sorbitol medium - Recipe for the production of 5% sorbitol medium.
Sour Dough Medium - Recipe for the preparation of sour dough medium.
Starch - Mineral Salt (STMS) Agar - Recipe for starch - mineral salt (STMS) agar.
Styrene Mineral Salts Medium - Recipe for Styrene Mineral Salts medium.
Terrific broth - Recipe for the preparation of terrific broth bacterial media.
Therinus Agar - Recipe for agar appropriate for he growth of Thermus genus
Thermus Medium - Recipe for media appropriate for the growth of Therums genus
Thiobacillus Medium F2 - Recipe for the production of Thiobacillus medium F2
Tomato Juice Agar - Recipe for the preparation of tomato juice agar,
Tomato Juice Medium - Recipe for the preparation of tomato juice medium,
Tomato Juice Yeast Extract Agar - Recipe for the preparation of tomato juice yeast extract agar.
Tomato Juice Yeast Extract Medium - Recipe for the preparation of tomato juice yeast extract
medium.
TSY agar - Trypticase soy yeast agar Recipe.
TSY broth - Trypticase soy yeast broth Recipe.
TYG Medium - Tryptone, yeast. glucose bacterial growth medium.
TYX Medium - Tryptone, yeast xylose bacterial growth medium.
Urea Medium - Recipe for the preparation of urea medium
Uric Acid Medium - Recipe for the preparation of uric acid medium
Whey Agar - Recipe for the preparation of whey agar.
Whey Medium - Recipe for the preparation of whey medium.
Wickerham Salt Agar - Recipe for -Wickerham Salt Agar.
Wickerham Salt Medium - Recipe for Wickerham Salt Medium.
Yeast Extract Glucose Medium - Yeast Extract Glucose medium recipe
YEL Agar - Recipe for YEL Agar.
YMF agar recipe - Recipe for preparation of YMF agar.
YMF medium recipe - Recipe for preparation of YMF medium.
YMG agar - Recipe for yeast and malt extract with glucose agar.
This agar is used for a number of Streptomyces species,
YMG media - Recipe for yeast and malt extract with glucose media. This media is used for a
number of Streptomyces species.
YPD Agar - Yeast extract/peptone/dextrose bacterial agar.
YPD media - Yeast extract/peptone/dextrose bacterial media.
YPG media - Yeast extract/peptone/galactose bacterial media,
YPM Agar - Recipe for YPM agar.
YPM Medium - Recipe for YPM medium.
YT (2x) - Yeast extract/tryptone bacterial media.

In a particular embodiment, the medium used for co-culturing a beneficial bacteria with Bacillus bacteria comprises manganese. In another embodiment, the medium comprises dextrose. In still another embodiment, the medium used for co-culturing comprises both manganese and dextrose.

Exemplary solid surfaces on which the culturing can be carried out include a wide range of substrates, ranging from various polymeric materials (silicone, polystyrene, polyurethane, and epoxy resins) to metals and metal oxides (silicon, titanium, aluminum, silica, and gold). Fabrication techniques (soft lithography and double casting molding techniques, microcontact printing, electron beam lithography, nanoimprint lithography, photolithography, electrodeposition methods, etc.)can be carried out on such materials in order to alter the topography of the solid surface.

Conditions of culturing or co-culturing may be adapted to enhance propagation of the bacteria and/or to enhance generation of the biofilm and/or to enhance incorporation of the beneficial bacteria into the biofilm producing bacteria. Such conditions include, but are not limited to environmental parameters such as pH, nutrient concentration, the ratio between the beneficial bacteria: biofilm producing bacteria and temperature.

In one embodiment, the culturing or co-culturing is carried out in a bioreactor.

As used herein, the term “bioreactor” refers to an apparatus adapted to support culturing of the beneficial bacteria.

The bioreactor may be adapted to support generation of a biofilm. In this case, the bioreactor will generally comprise one or more supports for the biofilm which may form a film thereover, and wherein the support is adapted to provide a significant surface area to enhance the formation of the biofilm. The bioreactors of the invention may be adapted for continuous throughput.

It will be appreciated that when a biofilm is generated in a bioreactor system, the conditions of the co-culture can be altered by altering the microfluidics (e.g. sheer stress) of the system.

The culturing (or co-culturing) of this aspect of the present invention may be carried out in the presence of additional agents that serve to increase propagation of the bacteria and/or enhance biofilm formation. Such agents include for example acetoin.

The amount of acetoin and the timing of addition may be altered so as to promote optimal biofilm production. In one embodiment, about 0.01-5% acetoin is used. In another embodiment, about 0.01-4% acetoin is used. In another embodiment, about 0.01-3% acetoin is used. In another embodiment, about 0.01-2% acetoin is used. In another embodiment, about 0.01-1% acetoin is used. In another embodiment, about 0.01-0.5% acetoin is used.

Co-cultures may be propagated for a length of time sufficient to generate a biofilm which incorporates both the beneficial bacteria and the biofilm generating bacteria.

According to one embodiment the co-cultures are grown to maximal plateau growth phase of the beneficial bacteria, at which time they may be harvested for maximal biofilm production.

According to another embodiment the co-cultures are grown to maximal plateau growth phase of the biofilm-producing bacteria, at which time they may be harvested for maximal biofilm production.

Thus, the bacteria may be cultured for at least 3 hours, at least 6 hours, at least 12 hours, at least 24 hours, 2 days, 3 days, 4 days, 5 days, 6 days or 7 days or longer. In one embodiment, the bacteria are not cultured for longer than 1 week, 2 weeks, 3 weeks, 4 weeks, 5 weeks or 6 weeks.

Once sufficient quantities of beneficial bacteria are propagated (and optionally encapsulated in the biofilm), the bacteria (or biofilm itself) may be harvested (i.e. removed from the growth substrate).

Following isolation from the growth substrate, the biofilm (and/or bacteria incorporated therein) may be subject to drying (i.e. dehydrating), freezing, spray drying, or freeze-drying. Preferably, the biofilm is treated in a way that preserves the viability of the bacteria.

According to one embodiment, the probiotic bacteria are formulated for topical delivery. Examples of topical formulations include but are not limited to those disclosed in US Patent Application No. 2015027296 and. US 20150182586, the contents of which are incorporated herein by reference.

In one embodiment, the compositions of probiotic bacteria described herein comprise no more than 100 species of bacteria, no more than 90 species of bacteria, no more than 80 species of bacteria, no more than 70 species of bacteria, no more than 60 species of bacteria, no more than 50 species of bacteria, no more than 40 species of bacteria, no more than 30 species of bacteria, no more than 20 species of bacteria, no more than 10 species of bacteria, no more than 5 species of bacteria, no more than 4 species of bacteria, no more than 3 species of bacteria and even no more than 2 species of bacteria.

It will be appreciated that the agents comprised in the topical formulation should be selected such that they do not compromise the viability of the bacterial populations within. The present inventors further contemplate using polymeric substance(s) or micelle(s), which can temporarily immobilize bacterial cells and provide partial protection to them.

In one embodiment, the topical formulation comprises a thick cream or emollient (made with viscosity control agents), film, polymer, latex and the like. The topical formulation may further comprise a nonionic surfactant may help further enhance the barrier properties of the composition, in addition to contributing to surface wetting. Examples of such surfactants may include, without limitation, polyoxyethylene-polyoxypropylene glycol (marketed as Pluronic F108). Another commonly used barrier agent is marketed as Pluronic P105. A latex material that provides an effective covering of the teat is described in U.S. Pat. No. 4,113,854. Suitable barrier forming agents include, for example, latex, arabinoxylanes, glucomannanes, guar gum, johannistree gums, cellulose, methyl cellulose, ethyl cellulose, hydroxyethyl cellulose, hydroxymethyl cellulose, carboxyethyl cellulose, carboxymethyl cellulose, starch, hydroxyethyl starch, gum arabic, curdlan, pullulan, dextran, polysuifonic acid, polyacryl amide, high molecular weight polyacrylate, high molecular weight cross-linked polyacrylate, carbomer, glycerol, sodium alginate, sodium alginate cross-linked with calcium salt, xanthan gum, polyvinyl alcohol) (PVA) and poly(N-vinylpyrrolidone) (PVP). Preferred embodiments for barrier-forming agents include xanthan gum, carboxymethyl cellulose, sodium alginate, sodium alginate cross-linked with calcium salt, PVA, hydroxyethyl cellulose, PVP, and (2,5-dioxo-4-imidazolidinyl)-urea (Allantoin).

A foaming agent may be used in the disclosed probiotic compositions. A foaming agent aerates a liquid composition to produce a foam that may increase surface area of the composition and improve contact with the surface to be treated (e.g., an animal teat). Typically, a foaming agent is in the form of a compressed gas, or a material that will decompose to release gas under certain conditions. Suitable gases include but are not limited to nitrogen, argon, air, carbon dioxide, helium and mixtures thereof. In addition, solid carbon dioxide (dry ice), liquid nitrogen, hydrogen peroxide and other substances that release gas via a change in state or through decomposition are contemplated for use with the present compositions. Typically, a high foaming surfactant such as sodium lauryl sulfate, dodecylbenzene sulfonic acid, sodium alkylaryl polyether sulfate, sodium lauryl ether sulfate, sodium decyl sulfate, cocamine oxide, C₁₂-C₁₄ whole coconut amido betaines can be used to generate a stable foam. The foam is produced when agitation in the form of a compressed gas is mixed with the solution either by bubbling the gas into the solution or spraying the solution or solution-gas mixture through spray equipment. Suitable gases include but are not limited to nitrogen, air, carbon dioxide and mixtures thereof. Foam can also be generated by the mechanical action of animals walking through the composition, or by other mechanical means that mix atmospheric air with the composition. The composition can be applied by having animals walk through an area containing the foam or by having the animal walk through a footbath solution that has foam floating on top of the solution.

Surfactants are well known for foaming and are widely used as foaming agents in hand soap and manual/hand dishwashing detergents and such surfactants can be used as foaming agents in applications where foaming can boost the performance and increase contact time of the composition to particular substrates. Examples of such. Suitable anionic surfactants can be chosen from a linear alkyl benzene sulfonic acid, a linear alkyl benzene sulfonate, an alkyl .alpha.-sulfomethyl ester, an .alpha.-olefin sulfonate, an alcohol ether sulfate, an alkyl sulfate, an alkylsulfo succinate, a dialkylsulfo succinate, and alkali metal, alkaline earth metal, amine and ammonium salts thereof. Specific examples are linear C₁₀-C₁₆ alkyl benzene sulfonic acid, linear C₁₀-C₁₆ alkyl benzene sulfonate or alkali metal, alkaline earth metal, amine and ammonium salt thereof e.g. sodium dodecylbenzenc sulfonate, sodium C₁₄-C₁₆α-olefin sulfonate, sodium methyl .alpha.-sulfomethyl ester and disodium methyl α-sulfo fatty acid salt. Suitable nonionic surfactants can be chosen from an alkyl polyglucoside, an alkyl ethoxylated alcohol, an alkyl propoxylated alcohol, an ethoxylatedpropoxylated alcohol, sorbitan, sorbitan ester, an alkanol amide. Specific examples include C₈-C₁₆ alkyl polyglucoside with a degree of polymerization ranging from 1 to 3 e.g., C₈-C₁₀ alkyl polyglucoside with a degree of polymerization of 1.5 (Glucopon® 200), C₈-C₁₆ alkyl polyglucoside with a degree of polymerization of 1.45 (Glucopon® 425), C₁₂-C₁₆ alkyl polyglucoside with a degree of polymerization of 1.6 (Glucopon® 625). Amphoteric surfactants can be chosen from alkyl betaines and alkyl amphoacetates. Suitable betaines include cocoamidopropyl betaine, and suitable amphoacetates include sodium cocoamphoacetate, sodium lauroamphoacetate and sodium cocoamphodiacetate, Alkyl amine oxides based on C12-C14 alkyl chain length feedstock such as those derived from coconut oil, palm kernel oil is also suitable foaming agents.

Viscosity control agents may be added to formulate the microbial compositions according to an intended environment of use. In one example, it is advantageous for some compositions to have an optimized solution viscosity to impart vertical clinging of the product onto a teat. This type of viscous product, especially one having a suitable thixotropic, pseudoplastic or viscoelastic gel strength, minimizes dripping of the product to avoid wastage and is particularly advantageous in teat dip compositions. Teat dip compositions may benefit from a preferred dynamic viscosity ranging from 1 cPs to 3000 cPs. Other applications including hard surface disinfectants have a preferred dynamic viscosity ranging from about 1 cPs to 300 cPs. The viscosity referred to throughout this application is Brookfield viscosity measured in cPs by a Brookfield LV viscometer at ambient temperature (25 degrees C.) with a spindle #2@3 to 30 rpm. In various embodiments, a thickener may be added to achieve a viscosity range of from 50 cPs to 10000 cPs, or from 100 cPs to 4000 cPs.

Suitable viscosity control agents include hemicellulose, for example arabinoxylanes and glucomannanes; plant gum materials, for example guar gum and johannistree gums; cellulose and derivatives thereof, for example methyl cellulose, ethyl cellulose, hydroxypropyl cellulose, hydroxyethyl cellulose or carboxymethyl cellulose; starch and starch derivatives, for example hydroxyethyl starch or cross linked starch; microbial polysaccharides, for example xanthan gum, sea weed polysaccharides, for example sodium alginate, carrageenan, curdlan, pullulan or dextran, dextran sulfate, whey, gelatin, chitosan, chitosan derivatives, polysulfonic acids and their salts, polyacrylamide, and glycerol. Preferred viscosity controlling agents are, different types of cellulose and derivatives thereof, particularly hydroxyalkyl cellulose, methyl cellulose, and glycerol. High molecular weight (MW>1,000,000) cross-linked polyacrylic acid type thickening agents are the products sold by B.F. Goodrich (now Lubrizol) under their Carbopol® trademark, especially Carbopol® 941, which is the most ion-insensitive of this class of polymers, and Carbopol® 940 and Carbopol® 934. The Carbopol® resins, also known as “Carbomer”, are reported in U.S. Pat. No. 5,225,096, and are hydrophilic high molecular weight, cross-linked acrylic acid polymers. Carbopol® 941 has a molecular weight of about 1,250,000, Carbopol® 940 has a molecular weight of approximately 4,000,000, and Carbopol 934 has a molecular weight of approximately 3,000,000. The Carbopol® resins are cross-linked with polyalkenyl polyether, e.g. about 1% of a polyallyl ether of sucrose having an average of about 5.8 allyl groups for each molecule of sucrose. Further detailed information on the Carbopol® resins is available from B.F. Goodrich (Lubrizol), see for example, the B. F. Goodrich catalog GC-67, Carbopol® Water Soluble Resins. Clays and modified clays such as bentonite or laponite can also be used as thickeners. Co-thickeners are often added to improve the stability of the gel matrix, for example, colloidal alumina or silica, fatty acids or their salts may improve gel stability. Typical viscosity control ingredients include xanthan gum, carboxymethyl cellulose, sodium alginate, sodium alginate cross-linked with calcium salt, polysulfonic acids and their salts, polyacrylamide, polyvinyl alcohol (PVA), hydroxyethyl cellulose and polyN-vinylpyrrolidone) (PVP).

Buffering and pH Adjusting Agents: A composition pH value may be selectively adjusted by the addition of acidic or basic ingredients, In one embodiment, an acidic pH is preferred. Suitable acids for use as pH adjusting agents may include, for example, citric acid, acetic acid, lactic acid, phosphoric acid, phosphorous acid, sulfamic acid, nitric acid, nitrous acid and hydrochloric acid. Mineral acids may be used to drastically lower the pH. The pH may be raised or made more alkaline by addition of an alkaline agent such as sodium hydroxide, potassium hydroxide, sodium carbonate, sodium bicarbonate, monosodium acid diphosphonate or combinations thereof. Traditional acid buffering agents such as citric acid, lactic acid, phosphoric acid may also be used to maintain a desired pH. The pH value of the composition may be adjusted by the addition of acidic or basic or buffering materials.

The physical property of pH may be adjusted by acid or base addition, and is broadly preferred in the range of from 2.0 to 8.0 for use in teat dip compositions and other compositions that are intended to contact the skin. In a more preferred sense this range is from 2.0 to 5.0, and a still more preferred range is from 2.5 to 4.5. Hard surface and commercial disinfectants may be provided with lower pH values, such as 2.0 or 1.0.

Wetting agent(s) or surface active agent(s) are also known as surfactants. Typical wetting agents are used to wet the surface of application, reduce surface tension of the surface of application so that the product can penetrate easily on the surface and remove unwanted soil. The wetting agents or surfactants of the composition increase overall detergency of the formula, solubilize or emulsify some of the organic ingredients that otherwise would not dissolve or emulsify, and facilitate penetration of active ingredients deep onto the surface of the intended application surfaces, such as teat skin.

Suitably effective surfactants used for wetting may include anionic, cationic, nonionic, zwitterionic and amphoteric surfactants. Wetting agents and surfactants used in the inventive applications can be high foaming, low foaming and non-foaming type. Suitable anionic surfactants can be chosen from a linear alkyl benzene sulfonic acid, a linear alkyl benzene sulfonate, an alkyl α-sulfomethyl ester, an α-olefin sulfonate, an alcohol ether sulfate, an alkyl sulfate, an alkylsulfo succinate, a dialkylsulfo succinate, and alkali metal, alkaline earth metal, amine and ammonium salts thereof. Specific examples are linear C₁₀-C₁₆ alkyl benzene sulfonic acid, linear C₁₀-C₁₆ alkyl benzene sulfonate or alkali metal, alkaline earth metal, amine and ammonium salt thereof e.g. sodium dodecylbenzene sulfonate, sodium C₁₀-C₁₆ α-olefin sulfonate, sodium methyl .alpha.-sulfomethyl ester and disodium methyl α-sulfa fatty acid salt. Suitable nonionic surfactants can be chosen from an alkyl polyglucoside, an alkyl ethoxylated alcohol, an alkyl propoxylated alcohol, an ethoxylatedpropoxylated alcohol, sorbitan, sorbitan ester, an alkanol amide. Specific examples include C₈-C₁₆ alkyl polyglucoside with a degree of polymerization ranging from 1 to 3 e.g., C₈-C₁₀ alkyl polyglucoside with a degree of polymerization of 1.5 (Glucopon® 200), C₈-C₁₆ alkyl polyglucoside with a degree of polymerization of 1.45 (Glucopon® 425), C₁₂-C₅ alkyl polyglucoside with a degree of polymerization of 1.6 (Glucopon® 625), and polyethoxylated polyoxypropylene block copolymers (poloxamers) including by way of example the Pluronic® poloxamers commercialized by BASF Chemical Co. Amphoteric surfactants can be chosen from alkyl betaines and alkyl amphoacetates. Suitable betaines include cocoamidopropyl betaine, and suitable amphoacetates include sodium cocoamphoacetate, sodium lauroamphoacetate and sodium cocoamphodiacetate.

It will be recognizable to those skilled in the art that because at least one surfactant (e.g., an anionic surfactant) is included as a synergistic microbial agent in this composition, that these surfactants would also have an influence on the wetting properties of the mixture.

Skin Conditioning Agents: Skin conditioning agents may also be optionally used in the disclosed compositions. Skin conditioning agents may provide extra protection for human or animal skin prior to or subsequent to being exposed to adverse conditions. For example, skin conditioning agents may include moisturizers, such as glycerin, sorbitol, propylene glycol, D-Panthenol, Poly Ethylene Glycol (PEG) 200-10,000, Poly Ethylene Glycol Esters, Acyl Lactylates, Polyquatemium-7, Glycerol Cocoate/Laurate, PEG-7 Glycerol Cocoate, Stearic Acid, Hydrolyzed Silk Peptide, Silk Protein, Aloe Vera Gel, Guar Hydroxypropyltrimonium Chloride, Alkyl Poly Glucoside/Glyceryl Luarate, shea butter and coco butter; sunscreen agents, such as titanium dioxide, zinc oxide, octyl methoxycinnamate (OMC), 4-methylbenzylidene camphor (4-MBC), oxybenzone and homosalate; and itch-relief or numbing agents, such as aloe vera, calamine, mint, menthol, camphor, antihistamines, corticosteroids, benzocaine and paroxamine HCl.

Pharmaceutical Carriers: A typical carrier or matrix for a probiotic composition is deionized water, although one skilled in the art will readily understand that other solvents or compatible materials other than water may be used to achieve the effective concentrations of bacteria. In some embodiments, a composition may contain at least about 60% water and preferably at least about 70% water by weight based on the total weight of the composition. Propylene glycol, glycol ethers and/or alcohols can also be used as a carrier either alone or in combination with water.

The carrier may also contain nutrients which help the bacteria propagate and remain viable.

In one embodiment, at least one of the probiotic bacteria described herein is provided to the bovine animal in the form of a biofilm.

In another embodiment, at least two of the probiotic bacteria described herein are provided to the bovine animal in the form of a biofilm, whereby one of the probiotic bacteria (which typically alone is not capable of generating a biofilm) is encapsulated by the second probiotic bacteria (which typically alone is capable of generating the biofilm).

In still another embodiment, the probiotic bacteria described herein have not been pre-cultured to form a biofilm. Instead the probiotic bacteria are provided to the bovine animal prior to formation of the biofilm. The free, unrestrained probiotic bacteria may be applied topically to the teat/udder area of the bovine animal under conditions that the biofilm begins to generate only after application.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may is include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than I in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” (SRL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials And Methods

Raw Milk Sampling:

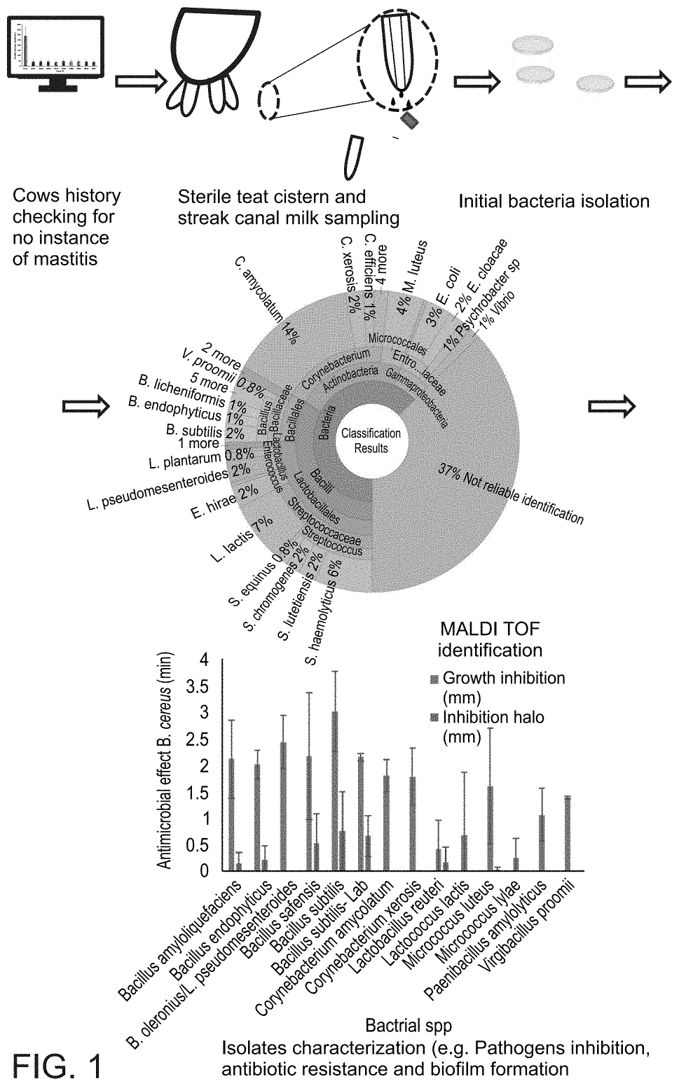

The schematic research plan is presented in FIG. 1. Nine Israeli Holstein dairy cows from the Israeli Agriculture Research Organization commercial dairy farm (Rishon LeZion, Israel) were chosen according to their history as was shown from the digital records of the herd book by NOA—The Israeli Dairy Herd Management Program (by the Israeli Cattle Breeders Association (ICBA)). Additional cows were used from two other dairies. These cows had at least two clean lactation cycles (and up to six). Clean lactation cycles were considered as those having less than 100 somatic cell counts (SCC) on their monthly milk quality tests across all the lactation period. The cows were sampled by hand milking in a sterile manner three times. The milking process was done as follows: (1) Cows were brought in near milking time to allow maximal microbial colonization time of the teat cistern population between milking periods, (2) cows udder teats (each quarter) were cleaned with a sterilizer cleaning cloth (company type) prior to the hand milking, (3) the three to six first milking hand strokes were collected into 15 ml sterile plastic tubes (volume of 3-6 ml of milk) as representative of the microbial teat cistern population, (4) additional milk samples were taken during the milking process to assess if any individual quarter presents mastitis or microbial infection indicators as described below. The raw milk samples were taken for further tests on the same day.

Bovine Clinical and Bacteriological Tests:

For bacteriological examination, 10 μl of each milk sample were inoculated onto blood agar (5% washed sheep red blood cells) and MacConkey agar plates (Bacto-Agar, Becton Dickinson). Plates were incubated at 37° C. and examined for bacterial growth after 18 and 42 h. To evaluate the status of the mammary gland immune reaction to infection, somatic cell differentiation was performed using flow cytometry (FACS Calibur flowcytometer, Becton-Dickinson) using anti-bovine monoclonal antibodies (VMRD) (Leitner et al., 2003). The monoclonal antibodies used were: anti-CD18/11a—BAT 75A (immunoglobulin [IgG-1]); anti-CD4—GC 50A1 138A (IgM); anti-CD8—CACT 80C (IgG-1); anti-CD21—BAQ 15A (IgM); anti-CD14—CAM 36A (IgG-1); and anti-polymorphonuclear (PMN) (G1) (IgM). All monoclonal antibodies used were species-reactive with bovine cells. Secondary polyclonal antibodies (Caltag Laboratories) used were goat anti-mouse IgG-1 conjugated with Tri-Color (TC) and goat anti-mouse IgM conjugated with FITC.

Initial Bacterial Isolation:

The initial teats' milk from the first sampling cycle was either concentrated by centrifugation (10000 rpm for 20 min) (centrifuge type) or plated directly from the samples. In the concentrated samples, the supernatant was discarded and the pellet was re-suspended in 250 μl of PBS. The concentrated samples and the non-concentrated samples were than plated on five different agar plates types: LB (Difco, Le pont de claix, France), Blood agar, PCA (toxoid, Basingstoke, England), BHI and MRS (Himedia, Mumbai, India) 10 μL for the concentrated samples, and 100 μL for the non-concentrated samples from each quarter (total of 36 samples in each cycle). The plates were incubated for a maximum of 72 hours. The isolates were taken from the incubator as soon as visible colonies were detected. The isolates were then compared with the bacteriological tests on the bulk milk samples. The isolates that were chosen for further testing were selected according to the colony morphology and the comparison with the bacteriological tests. The chosen colonies were mainly from those where the bulk milk cow samples were clean in the bacteriological test, but a significant number of colonies were observed in the initial milk samples. In total 140, 92 and 48 isolates were chosen from the first, second and third sampling cycle (respectively) and a glycerol stock was prepared and stored at −80° C.

MALDI TOF Analysis:

To further filter the isolates for relevant biotechnological relevant species, the isolates were identified by a Bruker Daltonik MALDI Biotyper (Bruker Daltonik GmbH, Bremen, Germany) classification analysis. The 280 isolates were prepared for analysis as follows: the selected colonies were suspended in 300 μl pure PCR grade water (Fisher, Australia) in 2 ml sterile plastic tubes and 900 μl of ethanol were added and after a short vortex centrifugation at 13,000 g for 2 min, the supernatant was discarded and the tubes were left to dry in a biological safety cabinet. Then 20 μl of 70% formic acid were added to the tubes and following vortexing, an additional 20 μl of pure acetonitrile was added to the tubes and mixed. The tubes were centrifuged at 13,000 g for 2 min. 1 μl of the supernatant was loaded into the MALDI Biotyper reading plate, dried and was overlaid with 1 μL of HCCA solution (Sigma-Aldrich, Rehovot, Israel). The plate was read in the Bruker Daltonik MALDI Biotyper and analyzed with the following MSP Libraries: BDAL, Mycobacteria Library (bead method), Filamentous Fungi, Listeria. The results were then transferred to a Krona visualization program²⁹.

Pathogen Inhibition Test:

For the antimicrobial characteristic test, isolates from the MALDI Biotyper classification analysis that were not known as either human pathogens or as causing mastitis in dairy cows were chosen. The isolates were plated on MRS or BHI plates according to their original source plates from the first isolation stage. The colonies from the plates were picked into 3 ml fresh liquid media again according to the plate media with one change, the MRS used was adjusted to pH 7 to prevent false inhibition by the lower pH of standard MRS media, and grown at 37° C. for 5 hours. In parallel, several common pathogenic bacterial species such as B. cereus ATCC10987, S. aureus ATCC and P. aeruginosa PA14 well as specific bovine pathogens: four coagulases negative staphylococcus (CNS) 171, 179, 188 and 189 and three general bovine pathogens 1989, 8944/2 and E. coli were prepared in the same way as the isolates. The microbial interaction test was performed on square BHI agar plates (BarNaor). 200 μL from the starter cultures of each pathogenic species as spread on the plates. A 5 mm circular Whatman 1 filter paper disks with 50 μl of the isolates starters were placed on the pathogen-plated surfaces. The plates were incubated O/N at 37° C. and then the inhibition zone (IZ) (the rca around the disk with no bacteria in it) and the isolate growth zone (GZ) (area with a clear distinctive growth morphology of the isolate around the disk) were measured and the inhibition index was calculated for each pathogen as follows:

$$Inhibition index = \frac{I {Z}_{j} + G {Z}_{j} + ( {IZ}_{j} \times G {Z}_{j} )}{\frac{1}{n} {\sum }_{j = 1}^{j = n} ( I {Z}_{j} + G {Z}_{j} + ( {IZ}_{j} \times G {Z}_{j} ) )} \qquad ( 1 )$$

j is a specific isolate and n is the total number of isolates tested for their antimicrobial properties.

The IZⱼ+GZⱼ+(IZⱼ×GZⱼ) equation was set to give more power to isolates that were shown to have IZ, as many isolates had an IZ=0.

Antibiotic Resistance Tests

All bacteria strains were grown on MRS or BHI hard agar plates according to their original source plates from the first isolation stage for 48 hours or overnight respectively, at 37° C. Then, the bacteria which grew on BHI plates were transferred to Mueller-Hinton broth agar plates (Himedia, Mumbai, India) and the bacteria which grew on MRS plates were transferred to the same medium plates. A standard disk agar diffusion Kirby-Bauer antibiotic testing³⁰ was performed with 10 relevant antibiotics for the dairy industry on 22 relevant isolates that were identified by the MALDI Biotyper. Their sensitivity and microbial inhibition concentrations (MIC) was measured in comparison to known bacterial species susceptibility.

Biofilm Formation Assay

All bacteria strains were grown on MRS or BHI hard agar plate for 48 h or overnight respectively, at 37° C. A starter culture of each strain was prepared using a single bacterial colony. Bacteria that were first grown on MRS were inoculated into 5 mL MRS broth for overnight and bacteria that were first grown on BHT medium were inoculated into 5 mL for 5 hours at 37° C.

Biofilm formation was identified by crystal violet staining similarly as described earlier (Assaf et al. 2015). Briefly, a 48-well polystyrene plate (Bar Naor) was seeded with 20 μl isolates culture, grown as described above with 180 μl liquid BHI or MRS media according to their original source plates from the first isolation stage for 24 hours or 48 hours.

The media was then discarded and the wells were washed three times with sterile distilled water and subsequently air-dried for 45 minutes. The wells were stained with 200 μl of 1% (v/v) crystal violet (Hylabs, Rehovot, Israel) for 15 minutes and washed twice with sterile distilled water. At this point, biofilms were visible as purple signs formed on the side of each well or in its bottom.

Statistical Analysis

Two- and one-way ANOVA and Tukey's Honest Significant Difference (HSD) post hoc test were used to compare means using a significance threshold of P=0.05. Statistical analyses were performed using JMP-IN software (version Pro 12, SAS, Cary, N.C.).

### Results

The present inventors focused on isolation and characterization of healthy teat cistern and streak canal microbiota. The assumption was that healthy cows that have never had severe mastitis might hold a more resilient microbiota in their udders. The goal was to create an effective pipeline (FIG. 1) for the isolation of relevant bacteria that may prevent the occurrence of mastitis and can be further used in future food and biotechnological applications.

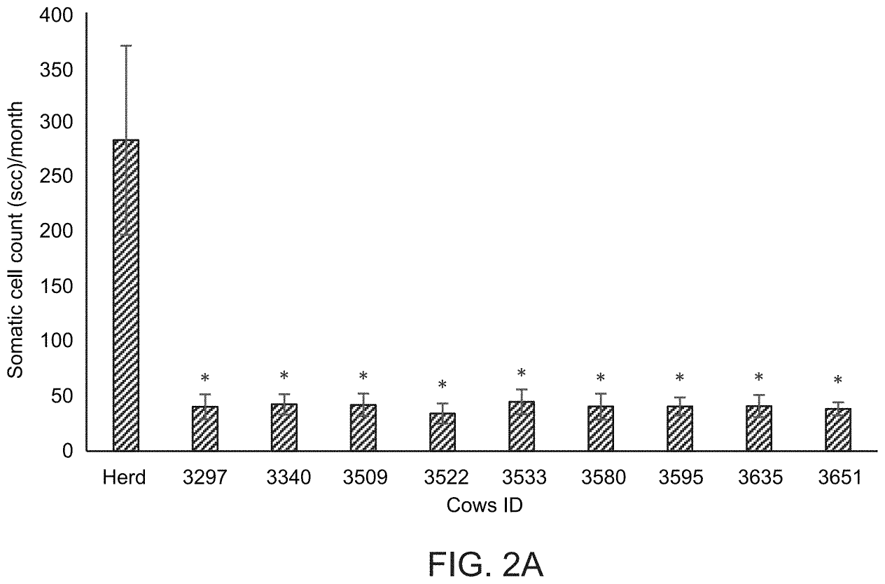

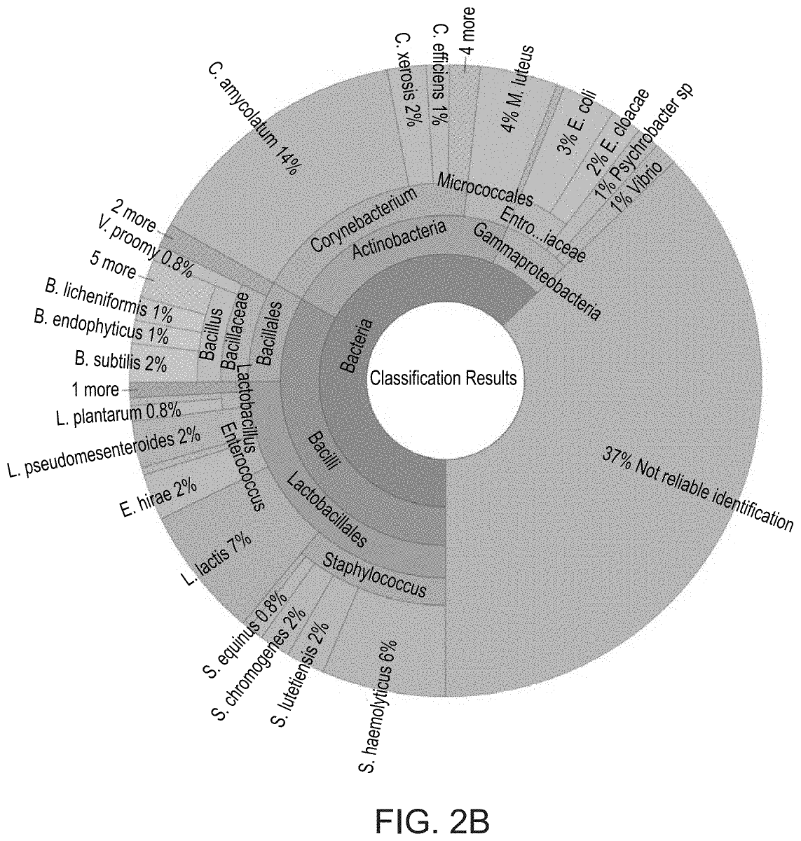

The first stage was to identify dairy cows that exhibit “healthy udders”. By using the computerized herd book with the NOA program, nine individual cows that has no mastitis events (average SCC<100) in at least two milking cycles, were chosen (FIG. 2A). In order to have the microbiota that characterize the streak canal, the sampling was taken from the foremilk¹¹ that is usually discarded because of fear of contamination.

Bacteria Isolation and Library Construction:

The foremilk samples were used for an initial isolation process of making a bacterial isolate library on typical agar isolation media^{8,11,17}. A total of 345 isolates were chosen according to their morphological characteristics and a comparison with immunological and bacteriological results that were taken from the same quarter. The criteria for choosing a specific isolate were: (1) coming from a quarter with no mastitis indicators in the bulk milk, (2) coming from a quarter with no severe bacteriological contamination in the bulk milk, (3) preferably coming from agar plates that showed large amounts of colonies yet the quarter was clean in the bulk milk samples.

MALDI Biotyper TOF Isolation Identification:

All 345 isolates were analyzed using a MALDI Biotyper TOF. As can be seen from FIG. 2B, 67% of the 253 isolates (from the Rishon dairy) were identified by the MALDI Biotyper and out of these 67%, 53% were recognized as Bacilli, 36% as Acitinobacteria and 11% as Gammaproteobacteria. From the Bacilli class, 75% are from the Lactobacillales and 25% are from Bacillales orders. In the Actinobacteria class, 81% are from the Corynebacterineae suborder, and 19% are from the Micrococcales order. From the Gammaproteobacteria class, 65% are from the Enterobacteriaceae order, 18% are from the Pseudomonadales order and 18% are from the Vibrionales order. Of the detected species, Corynebacterium amycolatum was identified as 14% of the total bacteria, Lactococcus lactis as 7%, and Staphylococcus haemolyticus as 6% of the total bacteria. The isolates that were chosen for further characterization were chosen as follows: (1) not known to cause bovine mastitis, (2) not known as a human pathogen and (3) may hold a probiotic characteristic. This elimination process resulted in the selection of 57 some of which were from the same taxa identification. The isolates included three of the Bacillus genera: B. subtilis, Bacillus sonorensis, Bacillus lichemformis, Bacilllus firmus, Bacillus megaterium, B. endophyticus and B. amyloliquefaciens, and also three of the Corynebacterium genera: C. minutissimum, C. amycolatum and C. xerosis. Other taxa that were isolated included L. lactis, L. pseudomesenteroides, Pedicoccus acidlilactici, M. luteus, L. plantarum, R. suwonensis and L. reuteri, some of which were recognized as probiotic taxa.

Characterization of Isolates Antimicrobial Relevant Properties Against a Pathogen Panel:

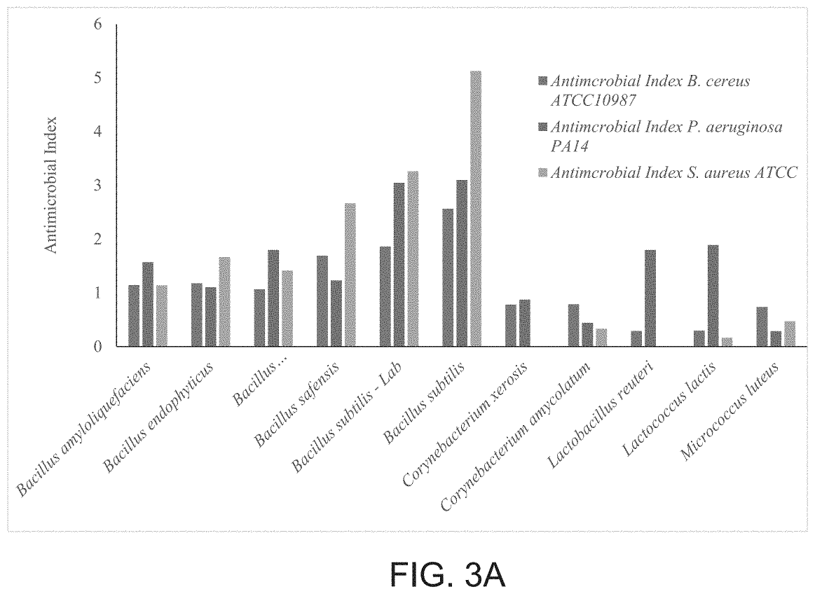

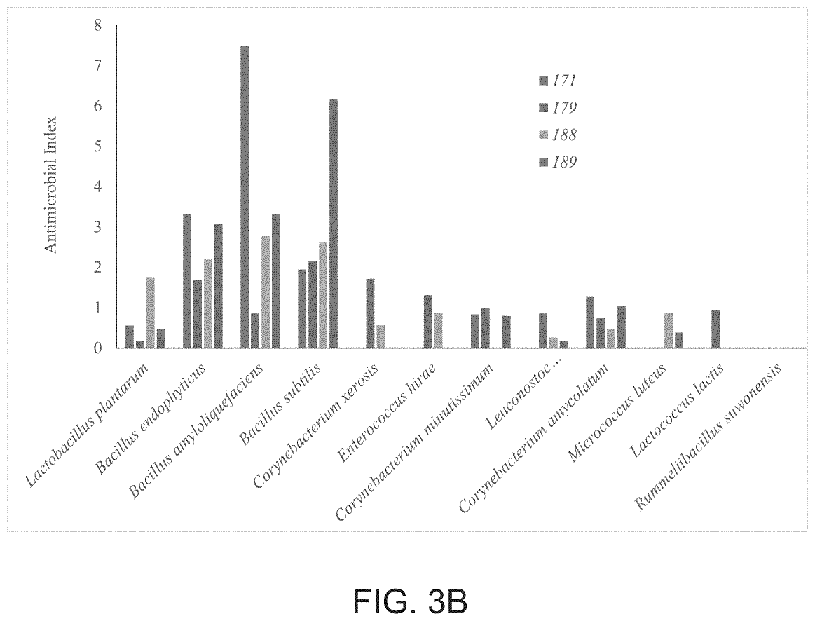

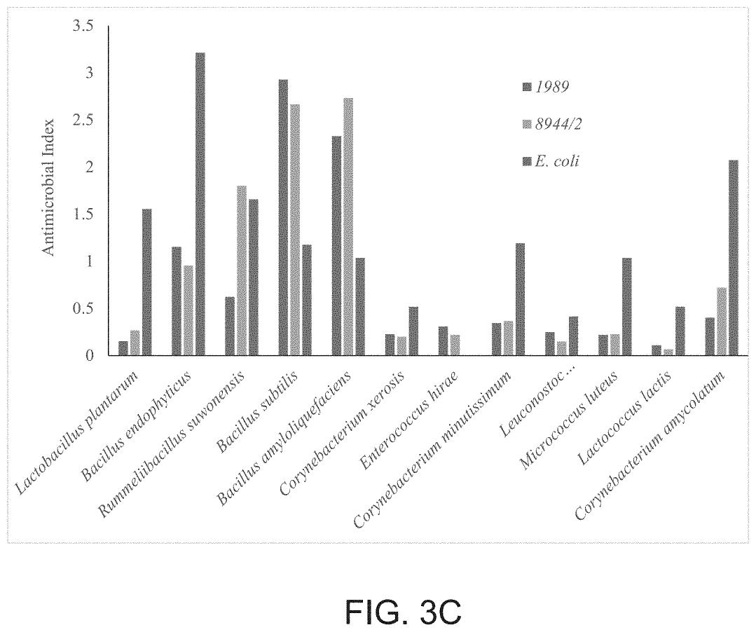

The selected 57 isolates from the MALDI Biotyper identification were tested for their ability to compete against pathogens in a resource abundant environment that can simulate the conditions on the cows' udders. The isolates and the pathogen were grown on rich media with no oxygen limitations and at 37° C., which is similar to the cows' body temperature. An antimicrobial index was formed (see the method section) in order to distinguish between the different isolates. The index gave more power to isolates shown to have an ability to produce antimicrobial materials. In the first stage, the isolates were tested against the general animal and food-borne bacterial pathogens such as Bacillus cereus, Pseudomonas aeruginosa and Staphylococcus aureus which represent broad spectrum and hard-to-treat pathogens^{19,20}(FIG. 3A). It was clear by the antimicrobial index that taxa from the Bacillus genera were the most potent against these general pathogens as B. subtilis has the highest index values. The several new B. subtilis strains from the isolation library showed higher antimicrobial activity than its parallel laboratory strain that also showed high index values. The lowest index values were of the M. luteus, C. xerosis and C. amycolatum taxa.

To further understand the potential of the isolates to serve as future sustainable agents against bovine mastitis, the isolates were tested against specific mastitis causing bacterial pathogens (FIGS. 3B and 3C). FIG. 3B describes the antimicrobial index of the isolates against four Coagulase Negative Staphylococcus (CNSs) isolated from individual cows with severe mastitis. Again, the Bacillus genera showed the highest antimicrobial activity and the strains with the highest activity were B. amyloliquefaciens and B. subtilis. FIG. 3C shows the isolates that were tested for their antimicrobial activity against three isolated pathogens from individual cows with severe mastitis: 1989, 8944/2 and E. coli. Again, the Bacillus genera were the most potent bacteria. Interestingly, C. amycolatum and R. suwonensis and L. plantarum had also relative high antimicrobial index values against E. coli.

Isolate Antibiotic Resistance and Biofilm Formation Tests:

The chosen isolates were tested by a standard diffusion method against a panel of 10 relevant antibiotics used in the dairy industry to classify their relative sensitivity (Table 1). From the antibiotic resistance test, the isolates from the strains L. lactis, R. suwonensis and B. amyloliquefaciens showed complete sensitivity to all the 10 antibiotics in the panel. The strains C. amycolatum and L. reuteri were sensitive to all the antibiotics except one (Trimethoprim-sulfamethoxazole and Lincomycin respectively). Sensitive taxa have an advantage in future biotechnological uses, as they present less danger as potentially virulent. The most resistant strain was an M. luteus isolate which was resistant to over four types of antibiotics that it was tested against (Table 1). The Bacillus genera were the dominant biofilm producers. Surprisingly, C. amycolatum was the only pellicle forming biofilm after 24 hours. The strains L. reuteri and L. lactis were the only strains to form a submerged biofilm.

16S rRNA Sequencing:

The 16S rRNA sequences of relevant bacteria identified as described herein is summarized in Tables 2 and 3.

TABLE 2

| ident | 16s | MALDI | TroF |
|---|---|---|---|
| 98% | L. Plantarum SEQ | L. Plantarum | B- 3297-1B S.3 L |
| | ID NOs: 1 and 2 | | |
| 99% | L. Plantarum | | |
| | SEQ ID NOs: 3 | L. Platarum | C- 3297-1A S.3 L |
| | and 4 | | |
| 97% | Corynebacterium | Corynebacterium | D- 3651-BB S.3 L |
| | amycolatum SEQ | amycolaturn | |
| | ID NO: 5 and 6 | | |
| 98% | Micrococcus | Micrococcus | E- 3522-4D |
| | luteus SEQ | luteus | |
| | ID NO: 7 and 8 | | |
| 99% | Lactococcus lactis | Lactococcus | F-3595-1C L |
| | SEQ ID NO: 9 | lactis | |
| 99% | Lactococcus lactis | Lactococcus | G-3533-1A L |
| | SEQ ID NO: 10 | lactis | |
| | and 11 | | |
| 98% | Bacillus subtilis | Bacillus subtilis | H- 3297-2C S.1 |
| | SEQ ID NO: 12 | | |
| | and 13 | | |
| 96% | Bacillus | Bacillus | I- 3297-2A S.1 |
| | endophyticus | endophyticus | |
| | SEQ ID NO: 14 | | |
| | and 15 | | |
| 99% | Bacillus subtilis | Bacillus subtilis | J- 3297-2C S.2 |
| | SEQ ID NO: 16 | | |
| | and 17 | | |
| 97% | Bacillus subtilis | Bacillus | K- 3297-2B |
| | SEQ ID NO: 18 | amyloliqefaciens | |
| | and 19 | | |
| 97% | Corynebacterium | Corynebacterium | M- 3522-1A S.3 L |
| | aurimucosum\ | minutissimum | |
| | Corynebactcrium | | |
| | nigricans | | |
| | SEQ ID NO: 20 | | |
| | and 21 | | |
| 98% | Bacillus subtilis | Bacillus | N-3297-2A S.2 |
| | SEQ ID NO: 22 | endophyticus | |
| | and 23 | | |

TABLE 3

| | | |
|---|---|---|
| Dairy Ortal 4 | Bacillus subtilis | SEQ ID NO: 24 |
| Dairy Ortal 26 | Pediococcus acidilactici | SEQ ID NO: 25 |
| Diary Ortal 27 | B. sonorensis\B.subtilis\ | SEQ ID NO: 26 |
| | B.lichentformis\B. haynesii | |
| Dairy Ortal 31 | Lactobacillus plantarum\ | SEQ ID NO: 27 |
| | Lactobacilllus pentosus | |
| Dairy Kalia 24 | Bacillus subtilis | SEQ ID NO: 28 |
| Dairy Kalia 27 | Bacillus subtilis | SEQ ID NO: 29 |
| Dairy Kalia 31 | Bacillus subtilis | SEQ ID NO: 30 |

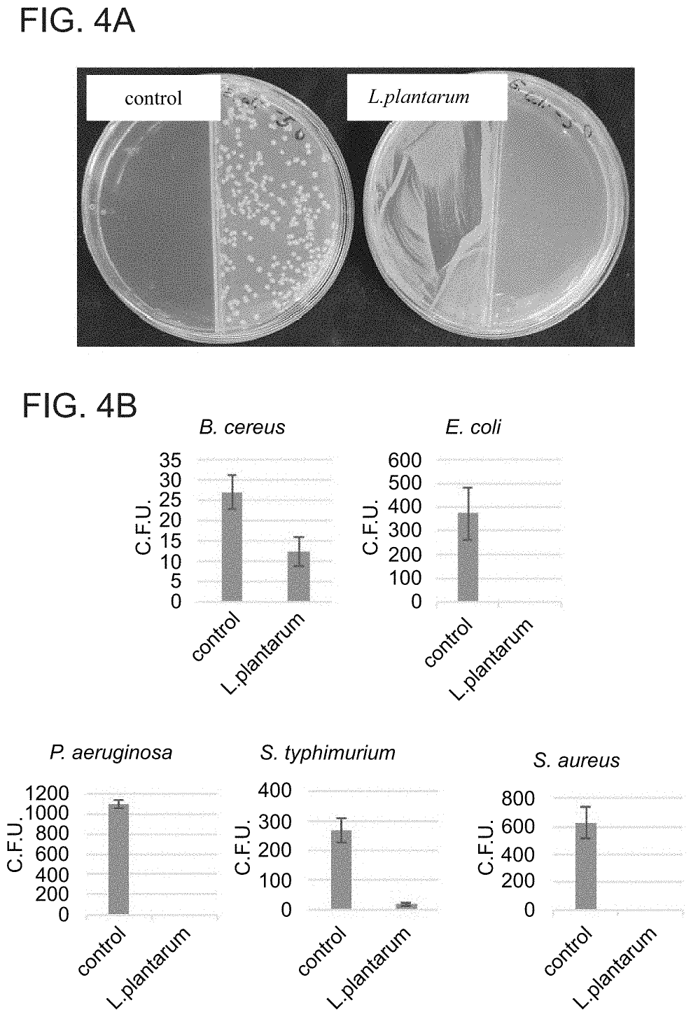

L. plantarum was plated on the MRS side of LB-MRS divided plates and incubated in aerobic conditions at 37° C. for 3 days. Then the LB side of the plate was incolated with various pathogens: B. cereus, E. coli, P. aeruginosa, typhimurium, and S. aureus. As can be seen in FIG. 4A-B, L. plantarum has a lethal effect of on these bacterial pathogens. Table 4 summarizes the volatile compounds that are secreted by L. plantarum.

TABLE 4

| VOC abundance (AU) | MRS | L. plantarum |
|---|---|---|
| acetic acid | 170.4 | 1802.4 |
| Butanal, 3-methyl- | 125.7 | 0.0 |
| 2-Butanone, 3-hydroxy- | 0.0 | 703.8 |
| Isoproturon | 95.3 | 84.5 |
| 2-Heptanone | 0.0 | 190.0 |
| Oxime-, methoxy-phenyl- | 0.0 | 46.0 |
| Pyrazine, 2,5-dimethyl- | 63.1 | 47.7 |
| Pyrazine, ethyl- | 132.4 | 104.6 |
| Benzaldehyde | 79.7 | 0.0 |
| Pyrazine, 2-ethy1-6-methyl- | 0.0 | 19.9 |
| 2-Nonanone | 0.0 | 161.7 |
| Nonanal | 82.8 | 0.0 |
| Adenosine, 5′-amino-5′-deoxy- | 0.0 | 47.0 |
| Benzothiazole | 36.0 | 26.1 |
| 2-Undecanone | 0.0 | 75.3 |
| 2-Tridecanone | 0.0 | 55.4 |
| 2-Pentadecanone | 0.0 | 57.3 |
| 2-Fleptadecanone | 0.0 | 31.9 |

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

### REFERENCES

1 Gomes, F. & Henriques, M. Control of bovine mastitis: old and recent therapeutic approaches. Current microbiology 72, 377-382 (2016).

2 Huijps, K., Lam, T. J. & Hogeveen, H. Costs of mastitis: facts and perception. Journal of Dairy Research 75, 113-120 (2008).

3 Hogeveen, H., Huijps, K. & Lam, T. Economic aspects of mastitis: new developments. New Zealand Veterinary Journal 59, 16-23 (2011).

4 Barlow, J. Mastitis therapy and antimicrobial susceptibility: a multispecies review with a focus on antibiotic treatment of mastitis in dairy cattle. Journal of mammary gland biology and neoplasia 16, 383-407 (2011).

5 Vanderhaeghen, W. et al. Identification, typing, ecology and epidemiology of coagulase negative staphylococci associated with ruminants. The Veterinary Journal 203, 44-51 (2015).

6 Jamali, H., Paydar, M., Radmehr, B., Ismail, S. & Dadrasnia, A. Prevalence and antimicrobial resistance of Staphylococcus aureus isolated from raw milk and dairy products. Food Control 54, 383-388 (2015).

7 Oliver, S. P., Boor, K. J., Murphy, S. C. & Murinda, S. E. Food safety hazards associated with consumption of raw milk. Foodborne pathogens and disease 6, 793-806 (2009).

8 Verdier-Metz, I. et ale Cow teat skin, a potential source of diverse microbial populations for cheese production. Applied and environmental microbiology 78, 326-333 (2012).

9 Al-Qumber, M. & Tagg, J. Commensal bacilli inhibitory to mastitis pathogens isolated from the udder microbiota of healthy cows. Journal of applied microbiology 101, 1152-1160 (2006).

10 Braem, G. et al. Antibacterial activities of coagulase-negative staphylococci from bovine teat apex skin and their inhibitory effect on mastitis-related patho gens. Journal of applied microbiology 116, 1084-1093 (2014).

11 Espeche, M. C., Otero, M. C., Sesma, F. & Nader-Macias, M. E. F. Screening of surface properties and antagonistic substances production by lactic acid bacteria isolated from the mammary gland of healthy and mastitic cows. Veterinary microbiology 135, 346-357 (2009).

12 Cui, X. et al. Antibacterial and Antibiofilm Activity of Lactic Acid Bacteria Isolated from Traditional Artisanal Milk Cheese from Northeast China Against Enteropathogenic Bacteria, Probiotics and antimicrobial proteins, 1-10 (2017).

13 Franciosi, E., Settanni, L., Cavazza, A. & Poznanski, E. Biodiversity and technological potential of wild lactic acid bacteria from raw cows' milk. International Dairy Journal 19, 3-11 (2009).

14 Innocente, N. et al. Effect of indigenous Lactobacillus rhamnosus isolated from bovine milk on microbiological characteristics and aromatic profile of traditional yogurt. LWT-Food Science and Technology 66, 158-164 (2016).

15 Pellegrino, M., Berardo, N., Giraudo, J., Nader-Macías, M. & Bogni, C. Bovine mastitis prevention: humoral and cellular response of dairy cows inoculated with lactic acid bacteria at the dry-off period. Beneficial microbes 8, 589-596 (2017).

16 Cho, I. & Blaser, M. J. The human microbiome: at the interface of health and disease. Nature Reviews Genetics 13, 260 (2012).

17 Normanno, G. et al. Occurrence, characterization and antimicrobial resistance of enterotoxigenic Staphylococcus aureus isolated from meat and dairy products. International journal of food microbiology 115, 290-296 (2007).

18 Ranadheera, R., Baines, S. & Adams, M. Importance of food in probiotic efficacy. Food Research International 43, 1-7 (2010).

19 Alekshun, M. N, & Levy, S. B. Molecular mechanisms of antibacterial multidrug resistance. Cell 128, 1037-1050 (2007).

20 Stenfors Arnesen, L. P., Fagerlund, A. & Gramm, P. E. From soil to gut: Bacillus cereus and its food poisoning toxins. FEMS microbiology reviews 32, 579-606 (2008).

21 Balcázar, J. L. & Rojas-Luna, T. Inhibitory activity of probiotic Bacillus subtilis UTM 126 against Vibrio species confers protection against vibriosis in juvenile shrimp (Litopenaeus vannamei). Current microbiology 55, 409-412 (2007).

22 Mora, I., Cabrefiga, J. & Montesinos, E. Antimicrobial peptide genes in Bacillus strains from plant environments. Int Microbiol 14, 213-223 (2011).

23 Capita, R. & Alonso-Calleja, C. Antibiotic-resistant bacteria: a challenge for the food industry. Critical reviews in food science and nutrition 53, 11-48 (2013).

24 Botrel, M.-A. et al. Distribution and antimicrobial resistance of clinical and subclinical mastitis pathogens in dairy cows in Rhône-Alpes, France. Foodborne pathogens and disease 7, 479-487 (2010),

25 Mathew, A. G., Cissell, R. & Liamthong, S. Antibiotic resistance in bacteria associated with food animals: a United States perspective of livestock production. Foodborne pathogens and disease 4, 115-133 (2007).

26 Shi, X. & Zhu, X. Biofilm formation and food safety in food industries. Trends in Food Science & Technology 20, 407-413 (2009).

27 Shemesh, M., Kolter, R. & Losick, R. The biocide chlorine dioxide stimulates biofilm formation in Bacillus subtilis by activation of the histidine kinase KinC. Journal of bacteriology 192, 6352-6356 (2010).

28 Pasvolsky, R., Zakin, V., Ostrova, I. & Shernesh, M. Butyric acid released during milk lipolysis triggers biofilm formation of Bacillus species. International journal of food microbiology 181, 19-27 (2014).

29 Ondov, B. D., Bergman, N. H. & Phillippy, A. M. Interactive metagenomic visualization in a Web browser, BMC bioinformatics 12, 385 (2011).

30 Bauer, A., Kirby, W., Sherris, J. C. & Turck, M. Antibiotic susceptibility testing by a standardized single disk method. American journal of clinical pathology 45, 493 (1966).

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, the priority document of this application is hereby incorporated herein by reference in its/their entirety.

1. A method of treating or preventing mastitis in a bovine animal comprising administering to the animal a therapeutically effective amount of at least one microbe selected from the group consisting of Bacillus subtilis, Bacillus sonorensis, Bacillus licheniformis, Bacilllus firmus, Bacillus megaterium, B. endophyticus, Bacillus safensis, Bacillus endophyticus, Bacillus amyloliquefaciens, Corynebacterium minutissimum, Corynebacterium amycolatum, Corynebacterium xerosis, Lactobacillus pentosus, Leuconostoc pseudomesenteroides, Micrococcus luteus, Rummeliibacillus suwonensis and Pediococcus acidilactici thereby treating or preventing mastitis in the animal.

2. The method of claim 1, wherein said at least one microbe is selected from the group consisting of Bacillus subtilis, Bacillus sonorensis, Bacillus licheniformis, Bacilllus firmus, Bacillus megaterium, B. endophyticus, Bacillus safensis, Bacillus endophyticus, Bacillus amyloliquefaciens, Lactobacillus pentosus, Leuconostoc pseudomesenteroides, Micrococcus luteus, Rummeliibacillus suwonensis and Pediococcus acidilactici.

3. The method of claim 1, wherein said at least one microbe is Bacillus subtilis.

4. A method of treating or preventing mastitis in a bovine animal comprising topically applying a therapeutically effective amount of a composition comprising at least one probiotic microbe and a biofilm-producing bacteria, under conditions that said probiotic microbe is encapsulated by said biofilm producing bacteria, to an udder of the animal thereby treating or preventing mastitis in the animal.

5-9. (canceled)

10. The method of claim 1, wherein said microbe is of the Bacillus genus.

11. The method of claim 4, wherein said biofilm-producing bacteria is of the Bacillus genus.

12. The method of claim 4, wherein said biofilm-producing bacteria is of a genus selected from the group consisting of C. amycolatum, L. reuteri and L. lactis.

13. The method of claim 4, wherein said probiotic is selected from the group consisting of Corynebacterium minutissimum, Corynebacterium xerosis, Leuconostoc pseudomesenteroides, Micrococcus luteus, Lactobacillus plantarum, Enterococcus hirae and Rummeliibacillus suwonensis.

14. The method of claim 1, wherein said mastitis is caused by a pathogen selected from the group consisting of S. aureus, E. coli and Coagulase Negative Staphylococcus (CNSs).

15. The method of claim 4, wherein said biofilm-producing bacteria encapsulates said probiotic bacteria prior to say applying.

16. The method of claim 4, wherein said biofilm-producing bacteria encapsulates said probiotic bacteria following said applying.

17. The method of claim 15, wherein said biofilm-producing bacteria encapsulates said probiotic bacteria by:
(a) in vitro co-culturing said probiotic bacteria with said biofilm-producing bacteria in a growth substrate under conditions that generate a biofilm which comprises said probiotic bacteria and biofilm-producing bacteria; and
(b) isolating said biofilm from said growth substrate.

18. The method of claim 17, wherein said growth substrate comprises a growth medium.

19. The method of claim 18, wherein said growth medium is selected from the group consisting of LB, LBGM, milk and MRS.

20-22. (canceled)

23. The method of claim 17, wherein said conditions comprise a pH of 6.8-7.5.

24. The method of claim 17, wherein said growth substrate comprises acetoin.

25. The method of claim 17, further comprising dehydrating said biofilm following said isolating.

26. The method of claim 4, wherein said probiotic microbe is a bacteria.

27. The method of claim 26, wherein said bacteria comprise no more than 50 bacterial species.

28. The method of claim 4, wherein said biofilm-producing bacteria are a single species of biofilm-producing bacteria.

29. A composition of matter comprising at least two species of bacteria of a genus selected from the group consisting of Bacillus subtilis, Bacillus sonorensis, Bacillus licheniformis, Bacilllus firmus, Bacillus megaterium, B. endophyticus, Bacillus endophyticus, Bacillus amyloliquefaciens, Corynebacterium minutissimum, Corynebacterium amycolatum, Corynebacterium xerosis, Lactococcus lactis, Leuconostoc pseudomesenteroides, Micrococcus luteus, Lactobacillus plantarum, Rummeliibacillus suwonensis and Lactobacillus reuteri, wherein the composition of matter is formulated for topical delivery.

30. The composition of claim 29, wherein said at least two species of bacteria are of a genus selected from the group consisting of Bacillus subtilis, Bacillus sonorensis, Bacillus licheniformis, Bacilllus firmus, Bacillus megaterium, B. endophyticus, Bacillus safensis, Bacillus endophyticus, Bacillus amyloliquefaciens, Lactobacillus pentosus, Lactococcus lactis, Leuconostoc pseudomesenteroides, Micrococcus luteus, Rummeliibacillus suwonensis, Lactobacillus plantarum and Pediococcus acidilactici.

31. The composition of claim 29, wherein one of said at least two species is Bacillus subtilis.

32. The composition of matter of claim 29, comprising no more than 50 species of bacteria.

33. The composition of matter of claim 29, wherein at least one of said at least two species produces a biofilm.

34. The composition of matter of claim 33, wherein said biofilm encapsulates the second of said at least two species.

35. (canceled)
